(19) United States
(12) Patent Application Publication
(10) Pub. No.: US 2020/0030459 A1
(43) Pub. Date: Jan. 30, 2020

(54) TARGETED DISRUPTION OF A CSF1-DAP12 PATHWAY MEMBER GENE FOR THE TREATMENT OF NEUROPATHIC PAIN

(71) Applicant: The Regents of the University of California, Oakland, CA (US)

(72) Inventors: Allan Basbaum, San Francisco, CA (US); Julia Kuhn, San Francisco, CA (US); Zhonghui Guan, San Francisco, CA (US)

(21) Appl. No.: 16/596,164

(22) Filed: Oct. 8, 2019

Related U.S. Application Data
(63) Continuation of application No. 15/516,872, filed on May 25, 2017.
(60) Provisional application No. 62/062,047, filed on Oct. 9, 2014.

(51) Int. Cl. A61K 48/00; C12N 15/113; C07K 14/535; C07K 14/705; C12N 9/22

(52) U.S. Cl. CPC A61K 48/0058; C12N 15/113; C07K 14/535; C07K 14/705; C12N 9/22; C12N 2830/008; C12N 2320/30; C12N 2320/32; C12N 2310/20; C12N 2799/021; A61K 48/0075

(57) ABSTRACT

The invention provides compositions and methods for treating neuropathic pain. Specifically, the disclosure provides a polynucleotide comprising a trigeminal ganglion (TGG) or dorsal root ganglion (DRG) promoter operably linked to a recombinant nucleic acid encoding an endonuclease that binds to a nucleotide sequence in the human colony stimulating factor 1 (hCSF1) gene and a method of using the polynucleotide or a vector comprising the polynucleotide for treatment of neuropathic pain.

### CROSS-REFERENCE TO RELATED APPLICATION

This application claims priority benefit of U.S. Provisional Application Ser. No. 62/062,047, filed Oct. 9, 2014, which application is incorporated herein by reference in its entirety.

### TECHNICAL FIELD

The invention relates generally to compositions and methods for the treatment of neuropathic pain.

### INTRODUCTION

Microglia contribute to many neurological conditions, including the mechanical hypersensitivity associated with neuropathic pain produced by peripheral nerve injury. There is little consensus, however, as to how nerve injury activates microglia. Activation of microglia requires an intact connection between the injured sensory neurons in dorsal root ganglia (DRG) and the spinal cord, injured DRG neurons must transmit signals that communicate with the microglia.

Several groups concluded that the chemokines, CCL2 and CCL21, provide the connection between injured sensory neurons and microglia. However, there is no expression of CCR2, the primary CCL2 receptor, in microglia, CCL2 cannot provide the connection between sensory neurons and spinal cord microglia. Also problematic is that under basal conditions microglia do not express the primary CCL21 receptor, CCR79. CCL21 does target CXCR3, but this receptor is expressed in microglia, astrocytes, and even neurons. Moreover, deletion of CXCR3 has no effect on nerve injury-induced hypersensitivity.

Accordingly, the components responsible for transmitting the signals between injured sensory neurons and microglia have yet to be elucidated.

### BRIEF SUMMARY

The present disclosure provides methods and compositions for treatment of neuropathic pain by targeted disruption of at least one CSF1-DAP12 pathway member gene (e.g., CSF1, DAP12) so as to effect a decrease in production of a CSF1-DAP12 pathway member. The present disclosure thus provides:

Feature 1. A polynucleotide comprising a neuronal promoter, such as a trigeminal ganglion (TGG) or dorsal root ganglion (DRG) promoter, operably linked to a recombinant nucleic acid encoding an endonuclease that binds to a nucleotide sequence of a CSF1-DAP12 pathway member, such as a colony stimulating factor 1 (CSF1) gene (e.g., human colony stimulating factor 1 (hCSF1) gene), a DAP12 gene (e.g., a human DAP12 (hDAP12) gene).

Feature 2. The polynucleotide of feature 1, wherein binding of the endonuclease to the nucleotide sequence in the decreases, reduces, or eliminated expression of at least one CSF1-DAP12 pathway member (e.g., hCSF and/or hDAP12) gene in a neuronal cell, such as a dorsal root ganglion cell.

Feature 3. The polynucleotide of features 1 or 2, wherein the neuronal promoter is a TGG or DRG promoter selected from the group consisting of: an hSYN1 promoter, a TRPV1 promoter, a Nav1.7 promoter, a Nav1.8 promoter, a Nav1.9 promoter, a CAG promoter, and an Advillin promoter.

Feature 4. The polynucleotide of any one of features 1-3, wherein the CSF1-DAP12 pathway member gene is nucleotide sequence in the hCSF1 gene is selected from the group consisting of: an hCSF1 gene regulatory region, an hCSF1 promoter, an hCSF1 transcription start site, an hCSF1 exon sequence, an hCSF1 intronic sequence, and an hCSF1 5′ or 3′ untranslated region.

Feature 5. The polynucleotide of any one of features 1-4, wherein the endonuclease is an endonuclease that is engineered to bind the nucleotide sequence of the CSF1-DAP12 pathway member gene (e.g., hCSF1 or hDAP12 gene).

Feature 6. The polynucleotide of feature 5, wherein the engineered endonuclease is a homing endonuclease, a transcription activator-like effector nucleases (TALENs), a zinc finger nuclease (ZFN), a Type II clustered regularly interspaced short palindromic repeats (CRISPR) associated (Cas9) nuclease, or a megaTAL nuclease.

Feature 7. The polynucleotide of feature 6, wherein the homing endonuclease is a LAGLIDADG endonuclease, a GIY-YIG endonuclease, a His-Cys box endonuclease, or an HNH endonuclease.

Feature 8. The polynucleotide of feature 6, wherein the homing endonuclease is I-Onu I, I HjeMI, I-CpaMI, I-Sce I, I-Chu I, I-Dmo I, I-Cre I, I-Csm I, PI-Sce I, PI-T11 I, PI-Mtu I, I-Ceu I, I-Sce II, I-Sce III, HO, P1-Civ I, PI-Ctr I, PI-Aae I, PI-Bsu I, PI-Dha I, PI-Dra I, PI-May I, PI-Mch I, PI-Mfu I, PI-Mfl I, PI-Mga I, PI-Mgo I, PI-Min I, PI-Mka I, PI-Mle I, PI-Mma I, PI-Msh I, PI-Msm I, PI-Mth I, PI-Mtu I, PI-Mxe I, PI-Npu I, PI-Pfu I, PI-Rma I, PI-Spb I, PI-Ssp I, PI-Fac I, PI-Mja I, PI-Pho I, PI-Tag I, PI-Thy I, PI-Tko I, or PI-Tsp I.

Feature 9. The polynucleotide of feature 6, wherein the Cas9 nuclease is from Streptococcus pyogenes, Streptococcus thermophilus, Treponema denticola, and Neisseria meningitidis.

Feature 10. The polynucleotide of feature 9, wherein the Cas9 nuclease comprises one or more mutations in a HNH or a RuvC-like endonuclease domain or the HNH and the RuvC-like endonuclease domains.

Feature 11. The polynucleotide of feature 10, wherein the mutant Cas9 nuclease is a nickase.

Feature 12. The polynucleotide of any one of the preceding features, wherein the polynucleotide further comprises a RNA polymerase III promoter operably linked to a crRNA and a tracrRNA, or to a single guide RNA (sgRNA).

Feature 13. The polynucleotide of feature 12, wherein the RNA polymerase III promoter is the human or mouse U6 snRNA promoter, the human or mouse H1 RNA promoter, or the human tRNA-val promoter.

Feature 14. The polynucleotide of feature 12, wherein the polynucleotide comprises a pair of offset crRNAs or sgRNAs.

Feature 15. The polynucleotide of any one of features 12-14, wherein the pair of crRNA or sgRNAs are offset by about 25 to about 100 nucleotides from each other.

Feature 16. The polynucleotide of any of the preceding features, wherein the endonuclease comprises a TREX2 domain.

Feature 17. A polynucleotide comprising a neuronal promoter, such as a promoter operable in a TGG or DRG that is operably linked to an inhibitory RNA that binds to an mRNA of a CSF1-DAP12 pathway member (e.g., an hCSF1 mRNA and/or a hDAP12 mRNA).

Feature 18. The polynucleotide of feature 17, wherein the neuronal promoter, e.g., TGG or DRG promoter, is an inducible promoter.

Feature 19. The polynucleotide of feature 18, wherein the inducible promoter comprises a tetracycline inducible promoter, a LOX-stop-LOX human or mouse U6 snRNA promoter, LOX-stop-LOX human or mouse H1 RNA promoter, or a LOX-stop-LOX human tRNA-val promoter.

Feature 20. The polynucleotide of feature 17, wherein the neuronal promoter, e.g., TGG or DRG promoter, is selected from the group consisting of: an hSYN1 promoter, a TRPV1 promoter, a Nav1.7 promoter, a Nav1.8 promoter, a Nav1.9 promoter, a CAG promoter, and an Advillin promoter.

Feature 21. The polynucleotide of feature 17, wherein the polynucleotide comprises a TGG or DRG promoter operably linked to a Cre recombinase and a LOX-stop-LOX inducible RNA polymerase III promoter operably linked to the inhibitory RNA.

Feature 22. The polynucleotide of any one of features 17-22, wherein the inhibitory RNA is an siRNA, an miRNA, an shRNA, a ribozyme, or a piRNA.

Feature 23. A vector comprising the polynucleotide of any one of features 1-22.

Feature 24. The vector of feature 23, wherein the vector is a plasmid-based vector or a viral vector.

Feature 25. The vector of feature 23 or feature 24, wherein the vector is episomal or non-integrative.

Feature 26. The vector of feature 25, wherein the viral vector is retroviral vector, an adenoviral vector, an adeno-associated viral (AAV) vector, or a herpes simplex virus (HSV) vector.

Feature 27. The vector of feature 26, wherein the retroviral vector is a lentiviral vector or a gamma retroviral vector.

Feature 28. The vector of feature 25, wherein the AAV comprises a serotype selected from the group consisting of: AAV9, AAV6, AAVrh10, AAV7M8, and AAV24YF.

Feature 29. The vector of feature 25, wherein the HSV vector comprises a serotype selected from the group consisting of: JΔNI5, JΔNI7, and JΔNI8.

Feature 30. A vector comprising a polynucleotide comprising an hSYN1 promoter operably linked to a nucleic acid encoding a Cas9 nuclease and a polynucleotide comprising an U6 RNA polymerase III promoter operably linked to CSF1-DAP12 pathway member gene targeted sgRNA (e.g., an hCSF1 gene targeted sgRNA or an hDAP12 gene targeted sgRNA).

Feature 31. A method of treating neuropathic or central pain comprising administering a subject in need thereof, a vector according to any one of features 17-30.

Feature 32. A method of providing analgesia to a subject comprising administering to the subject, a vector according to any one of features 17-30. Feature 33. A method of decreasing expression of at least one CSF1-DAP12 pathway member gene (e.g., an hCSF1 gene, an hDAP12 gene) in a neuron (e.g., TGG or DGG) of a subject, comprising administering to the subject, a vector according to any one of features 17-30.

Feature 34. A method of reducing nerve injury induced mechanical hypersensitivity and microglia activation comprising administering to the subject, a vector according to any one of features 17-30.

Feature 35. The method of any one of features 31-34, wherein the vector is administered to the subject by intrathecal bolus injection or infusion, intraganglionic injection, intraneural injection, subcutaneous injection, or intraventricular injection.

Feature 36. The method of features 31-35, wherein the vector is administered to the subject by intrathecal bolus injection or infusion at multiple levels of the spinal column for DRG transduction.

Feature 37. The method of features 31-35, wherein the vector is administered to the subject by intraganglionic injection directly into a single dorsal root ganglion, multiple dorsal root ganglia, or the trigeminal ganglion.

Feature 38. The method of features 31-35, wherein the vector is administered to the subject by intraneural injection into the nerve bundle (e.g. sciatic nerve, trigeminal nerve).

Feature 39. The method of features 31-35, wherein the vector is administered to the subject by subcutaneous injection at the peripheral nerve terminals (subdermal or internal organ wall).

Feature 40. The method of features 31-35, wherein the vector is administered to the subject by intraventricular injection (for trigeminal ganglion transduction).

Feature 41. The method of features 31-15, wherein the neuropathic pain is central neuropathic pain, and the vector is administered by intraparenchymal administration, intracisternal administration, intracranial administration, intraspinal administration or stereotactic brain injection.

### BRIEF DESCRIPTION OF THE SEVERAL VIEWS OF THE DRAWINGS

FIG. 1A-1O depict the effect of CSF1 induction in DRG neurons after peripheral nerve injury.

FIG. 2A-2H depict the effect of CSF1 induction in DRG neurons after peripheral nerve injury.

FIG. 3A-3D depict the requirement of DAP12 for nerve injury- and CSF1-induced mechanical hypersensitivity.

FIG. 4A-4E depict that in DAP12 ko mice baseline motor and pain behaviors are intact and SNI-induced de novo CSF1 expression in DRG neurons is preserved.

FIG. 5A-5G depict the requirement of DAP12 for nerve injury and CSF1-induced microglial gene induction, but not for microglia proliferation.

FIG. 6 depicts the contribution of DAP12 to the autotomy phenotype.

FIG. 7 depicts that microglia-enriched genes are induced in the dorsal cord after nerve injury; monocyte-specific genes are not.

FIG. 8A-8E depict that peripheral nerve injury induces microglial proliferation.

FIG. 9A-9D depict that nerve injury and CSF1-induced microglia proliferation in the dorsal horn is DAP12-independent.

FIG. 10A-10K depict that nerve injury-induced CSF1 expression in injured motoneurons is required for ventral horn microglial activation.

FIG. 11 depicts the coexpression of ATF3 and CSF1 in injured motoneurons.

FIG. 12A-12C depict that CSF1 is upregulated in injured motoneurons and is required for nerve injury-induced microglia activation and proliferation in the ventral horn of the spinal cord.

FIG. 13 depicts nerve injury-induced CSF1 in injured sensory neurons is preserved in Nestin-Cre; Csf1 fl/fl mice.

FIG. 14 depicts a schematic showing de novo expression of CSF1 in injured sensory neurons triggers a microglial, DAP12-dependent induction of genes that contribute to neuropathic pain.

FIG. 15 depicts a schematic showing de novo CSF1 expression in injured sensory neurons triggers a DAP12-independent self-renewal of microglia and a DAP12-dependent upregulation of microglial genes that contribute to the neuropathic pain phenotype.

FIG. 16A-16B depict the effect of minocycline on CSF1-induced hypersensitivity (FIG. 16A), and intrathecal CSF1-induced mechanical hypersensitivity in P2X4 mutant mice (FIG. 16B).

### DETAILED DESCRIPTION

The practice of the invention will employ, unless indicated specifically to the contrary, conventional methods of chemistry, biochemistry, organic chemistry, molecular biology, microbiology, recombinant DNA techniques, genetics, immunology, and cell biology that are within the skill of the art, many of which are described below for the purpose of illustration. Such techniques are explained fully in the literature. See, e.g., Sambrook, et al., Molecular Cloning: A Laboratory Manual (3rd Edition, 2001); Sambrook, et al., Molecular Cloning: A Laboratory Manual (2nd Edition, 1989); Maniatis et al., Molecular Cloning: A Laboratory Manual (1982); Ausubel et al., Current Protocols in Molecular Biology (John Wiley and Sons, updated July 2008); Short Protocols in Molecular Biology: A Compendium of Methods from Current Protocols in Molecular Biology, Greene Pub. Associates and Wiley-Interscience; Glover, DNA Cloning: A Practical Approach, vol. I & II (IRL Press, Oxford, 1985); Anand, Techniques for the Analysis of Complex Genomes, (Academic Press, New York, 1992); Transcription and Translation (B. Hames & S. Higgins, Eds., 1984); Perbal, A Practical Guide to Molecular Cloning (1984); and Harlow and Lane, Antibodies, (Cold Spring Harbor Laboratory Press, Cold Spring Harbor, N.Y., 1998).

All publications, patent applications, and issued patents cited in this specification are herein incorporated by reference as if each individual publication, patent application, or issued patent were specifically and individually indicated to be incorporated by reference.

Although the foregoing invention has been described in some detail by way of illustration and example for purposes of clarity of understanding, it will be readily apparent to one of ordinary skill in the art in light of the teachings of this invention that certain changes and modifications may be made thereto without departing from the spirit or scope of the appended claims. The following examples are provided by way of illustration only and not by way of limitation. Those of skill in the art will readily recognize a variety of noncritical parameters that could be changed or modified to yield essentially similar results.

Unless defined otherwise, all technical and scientific terms used herein have the same meaning as commonly understood by those of ordinary skill in the art to which the invention belongs. Although any methods and materials similar or equivalent to those described herein can be used in the practice or testing of the present invention, preferred embodiments of compositions, methods and materials are described herein. For the purposes of the present invention, the following terms are defined below.

The articles “a,” “an,” and “the” are used herein to refer to one or to more than one (i.e., to at least one) of the grammatical object of the article. By way of example, “an element” means one element or more than one element.

As used herein, the term “about” or “approximately” refers to a quantity, level, value, number, frequency, percentage, dimension, size, amount, weight or length that varies by as much as 30, 25, 20, 25, 10, 9, 8, 7, 6, 5, 4, 3, 2 or 1% to a reference quantity, level, value, number, frequency, percentage, dimension, size, amount, weight or length. In particular embodiments, the terms “about” or “approximately” when preceding a numerical value indicates the value plus or minus a range of 15%, 10%, 5%, or 1%.

Throughout this specification, unless the context requires otherwise, the words “comprise”, “comprises” and “comprising” will be understood to imply the inclusion of a stated step or element or group of steps or elements but not the exclusion of any other step or element or group of steps or elements. By “consisting of” is meant including, and limited to, whatever follows the phrase “consisting of.” Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that no other elements are optional and may or may not be present depending upon whether or not they affect the activity or action of the listed elements.

Reference throughout this specification to “one embodiment,” “an embodiment,” “a particular embodiment,” “a related embodiment,” “a certain embodiment,” “an additional embodiment,” or “a further embodiment” or combinations thereof means that a particular feature, structure or characteristic described in connection with the embodiment is included in at least one embodiment of the present invention. Thus, the appearances of the foregoing phrases in various places throughout this specification are not necessarily all referring to the same embodiment. Furthermore, the particular features, structures, or characteristics may be combined in any suitable manner in one or more embodiments.

In view of the findings discussed herein, targeting the CSF1-DAP12 pathway, e.g., to decrease expression of a CSF1-DAP12 pathway member (e.g., at least one of CSF1, CSFR1, or DAP12) thus provides a novel approach to the pharmacological management of neuropathic pain and potentially also to a host of peripheral nerve injury-induced alterations in motoneuron function.

The present disclosure thus provides compositions and methods to effect targeted disruption of at least one CSF1-DAP12 pathway member gene (e.g., CSF1, DAP12) for treatment or prevention of neuropathic pain in a subject having or at risk of neuropathic pain.

Further, since as evidenced herein there is peripheral as well as central transport of CSF1 that is induced after injury, CSF1 can play a role in recruitment of macrophages to the nerve damage (neuroma) site in the periphery. Thus, targeted disruption of CSF1 can also provide for a method of reducing recruitment of macrophages to a site of peripheral nerve damage by administering to a subject in need of treatment a modulator of a CSF1-DAP12 pathway member gene (e.g., CSF1 (e.g., hCSF1), DAP12, (e.g., hDAP12).

### Compositions for Modulating Expression of a CSF1-DAP12 Pathway Member

Compositions for treatment of neuropathic pain contemplate by the present disclosure include nucleic acid compositions to target at least one CSF1-DAP12 pathway member (e.g., CSF1, DAP12). Such compositions can, for example, effect inhibition of expression of a target gene by, for example, genome editing (e.g., to effectively delete all or a portion of a gene encoding the target gene), inhibiting production of RNA encoding a target protein, and/or inhibiting translation of RNA encoding a target protein. A variety of tools are available to make and use such compositions to effectively target a gene to decrease its expression as described herein. Furthermore, the sequences of mammalian CSF1-DAP12 pathway member genes are available in the art (e.g., human CSF-1, human DAP12), as are methods of adapting platforms to specifically target a gene or interest. Compositions for use in targeting at least one CSF1-DAP12 pathway member such as disclosed below may be referred to herein as CSF1-DAP12 pathway modulators.

In particular embodiments, polynucleotides are provided comprising a neuronal promoter, and may be a neuron-specific promoter. The promoter may be selected according to the type of neuron to be treated, e.g., for use in treatment of peripheral neuropathic pain or central neuropathic pain. For example, the neuronal promoter can be a trigeminal ganglion (TGG) or dorsal root ganglion (DRG) promoter operably linked to a recombinant nucleic acid encoding an endonuclease that binds to a nucleotide sequence in a CSF1-DAP12 pathyway member gene (e.g., a CSF1 gene, a human colony stimulating factor 1 (hCSF1) gene, a DAP12 gene, a human DAP12 gene).

Other examples of promoter sequences operable in a neuron include, but are not limited to, a neuron-specific enolase (NSE) promoter (see, e.g., EMBL HSENO2, X51956); an aromatic amino acid decarboxylase (AADC) promoter; a neurofilament promoter (see, e.g., GenBank HUMNFL, L04147); a synapsin promoter (see, e.g., GenBank HUMSYNIB, M55301); a thy-1 promoter (see, e.g., Chen et al. (1987) Cell 51:7-19); a serotonin receptor promoter (see, e.g., GenBank S62283); a tyrosine hydroxylase promoter (TH) (see, e.g., Nucl. Acids. Res. 15:2363-2384 (1987) and Neuron 6:583-594 (1991)); a GnRH promoter (see, e.g., Radovick et al., Proc. Natl. Acad. Sci. USA 88:3402-3406 (1991)); an L7 promoter (see, e.g., Oberdick et al., Science 248:223-226 (1990)); a DNMT promoter (see, e.g., Bartge et al., Proc. Natl. Acad. Sci. USA 85:3648-3652 (1988)); an enkephalin promoter (see, e.g., Comb et al., EMBO J. 17:3793-3805 (1988)); a myelin basic protein (MBP) promoter; and a CMV enhancer/platelet-derived growth factor-β promoter (see, e.g., Liu et al. (2004) Gene Therapy 11:52-60). Promoters operable in a neuron, including neuron-specific promoters and other control elements (e.g., enhancers), are known in the art.

As used herein, the terms “polynucleotide” or “nucleic acid” refer to deoxyribonucleic acid (DNA), ribonucleic acid (RNA) and DNA/RNA hybrids. Polynucleotides may be single-stranded or double-stranded. Polynucleotides include, but are not limited to: pre-messenger RNA (pre-mRNA), messenger RNA (mRNA), RNA, short interfering RNA (siRNA), short hairpin RNA (shRNA), microRNA (miRNA), ribozymes, synthetic RNA, genomic RNA (gRNA), plus strand RNA (RNA(+)), minus strand RNA (RNA(−)), tracrRNA, crRNA, single guide RNA (sgRNA), synthetic RNA, genomic DNA (gDNA), PCR amplified DNA, complementary DNA (cDNA), synthetic DNA, or recombinant DNA.

Polynucleotides refer to a polymeric form of nucleotides of at least 5, at least 10, at least 15, at least 20, at least 25, at least 30, at least 40, at least 50, at least 100, at least 200, at least 300, at least 400, at least 500, at least 1000, at least 5000, at least 10000, or at least 15000 or more nucleotides in length, either ribonucleotides or deoxynucleotides or a modified form of either type of nucleotide, as well as all intermediate lengths. It will be readily understood that “intermediate lengths,” in this context, means any length between the quoted values, such as 6, 7, 8, 9, etc., 101, 102, 103, etc.; 151, 152, 153, etc.; 201, 202, 203, etc. In particular embodiments, polynucleotides or variants have at least or about 50%, 55%, 60%, 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% sequence identity to a reference sequence described herein or known in the art, typically where the variant maintains at least one biological activity of the reference sequence.

An “isolated polynucleotide,” as used herein, refers to a polynucleotide that has been purified from the sequences which flank it in a naturally-occurring state, e.g., a DNA fragment that has been removed from the sequences that are normally adjacent to the fragment. In particular embodiments, an “isolated polynucleotide” refers to a complementary DNA (cDNA), a recombinant DNA, or other polynucleotide that does not exist in nature and that has been made by the hand of man.

Terms that describe the orientation of polynucleotides include: 5′ (normally the end of the polynucleotide having a free phosphate group) and 3′ (normally the end of the polynucleotide having a free hydroxyl (OH) group). Polynucleotide sequences can be annotated in the 5′ to 3′ orientation or the 3′ to 5′ orientation. For DNA and mRNA, the 5′ to 3′ strand is designated the “sense,” “plus,” or “coding” strand because its sequence is identical to the sequence of the premessenger (premRNA) [except for uracil (U) in RNA, instead of thymine (T) in DNA]. For DNA and mRNA, the complementary 3′ to 5′ strand which is the strand transcribed by the RNA polymerase is designated as “template,” “antisense,” “minus,” or “non-coding” strand. As used herein, the term “reverse orientation” refers to a 5′ to 3′ sequence written in the 3′ to 5′ orientation or a 3′ to 5′ sequence written in the 5′ to 3′ orientation.

The terms “complementary” and “complementarity” refer to polynucleotides (i.e., a sequence of nucleotides) related by the base-pairing rules. For example, the complementary strand of the DNA sequence 5′ AGTCATG 3′ is 3′ T C A G T A C 5′. The latter sequence is often written as the reverse complement with the 5′ end on the left and the 3′ end on the right, 5′ C A T G A C T 3′. A sequence that is equal to its reverse complement is said to be a palindromic sequence. Complementarity can be “partial,” in which only some of the nucleic acids' bases are matched according to the base pairing rules. Or, there can be “complete” or “total” complementarity between the nucleic acids.

As used herein, the term “gene” may refer to a polynucleotide sequence comprising enhancers, promoters, introns, exons, and the like. In particular embodiments, the term “gene” refers to a polynucleotide sequence encoding a polypeptide, regardless of whether the polynucleotide sequence is identical to the genomic sequence encoding the polypeptide.

A “genomic sequence regulating transcription of” or a “genomic sequence that regulates transcription or” refers to a polynucleotide sequence that is associated with the transcription of a gene.

In one embodiment, a polynucleotide-of-interest comprises an inhibitory polynucleotide including, but not limited to, a crRNA, a tracrRNA, a single guide RNA (sgRNA), an siRNA, an miRNA, an shRNA, piRNA, a ribozyme or another inhibitory RNA.

In one embodiment, a polynucleotide-of-interest comprises a crRNA, a tracrRNA, or a single guide RNA (sgRNA). These RNAs are part of the CRISPR (Clustered Regularly Interspaced Short Palindromic Repeats)/Cas (CRISPR Associated) nuclease system; a recently engineered nuclease system based on a bacterial system that can be used for mammalian genome engineering. See, e.g., Jinek et al. (2012) Science 337:816-821; Cong et al. (2013) Science 339:819-823; Mali et al. (2013) Science 339:823-826; Qi et al. (2013) Cell 152:1173-1183; Jinek et al. (2013), eLife 2:e00471; David Segal (2013) eLife 2:e00563; Ran et al. (2013) Nature Protocols 8(11):2281-2308; PCT Pub. Nos.: WO2007025097; WO2008021207; WO2010011961; WO2010054108; WO2010054154; WO2012054726; WO2012149470; WO2012164565; WO2013098244; WO2013126794; WO2013141680; WO2013142578; U.S. Pat. App. Pub. Nos: US20100093617; US20130011828; US20100257638; US20100076057; US20110217739; US20110300538; US20130288251; US20120277120; and U.S. Pat. No. 8,546,553, each of which is incorporated herein by reference in its entirety.

The CRISPR/Cas nuclease system can be used to introduce a double-strand break in a target polynucleotide sequence, which may be repaired by non-homologous end joining (NHEJ) in the absence of a polynucleotide template, e.g., a DNA template, or by homology directed repair (HDR), i.e., homologous recombination, in the presence of a polynucleotide repair template. Cas9 nucleases can also be engineered as nickases, which generate single-stranded DNA breaks that can be repaired using the cell's base-excision-repair (BER) machinery or homologous recombination in the presence of a repair template. NHEJ is an error-prone process that frequently results in the formation of small insertions and deletions that disrupt gene function. Homologous recombination requires homologous DNA as a template for repair and can be leveraged to create a limitless variety of modifications specified by the introduction of donor DNA containing the desired sequence flanked on either side by sequences bearing homology to the target. In one embodiment, wherein a crRNA or sgRNA is directed against a polynucleotide sequence encoding a polypeptide, NHEJ of the ends of the cleaved genomic sequence may result in a normal polypeptide, a loss-of- or gain-of-function polypeptide, or knock-out of a functional polypeptide. In another embodiment, wherein a crRNA or sgRNA is directed against a polynucleotide sequence encoding a cis-acting sequence that regulates mRNA expression of a polynucleotide sequence encoding a polypeptide, NHEJ of the genomic sequence may result increased expression, decreased expression, or complete loss of expression of the mRNA and polypeptide.

As used herein, the term “crRNA” refers to an RNA comprising a region of partial or total complementarity referred to herein as a “spacer motif” to a target polynucleotide sequence referred to herein as a protospacer motif. In one embodiment, a protospacer motif is a 20 nucleotide target sequence. In particular embodiments, the protospacer motif is 18, 19, 20, 21, 22, 23, 24, 25, 26, 27, 28, 29, 30, or more nucleotides. Without wishing to be bound by any particular theory, it is contemplated that protospacer target sequences of various lengths will be recognized by different bacterial species.

In one embodiment, the region of complementarity comprises a polynucleotide sequence that is at least 80%, 81%, 82%, 83%, 84%, 85%, 86%, 87%, 88%, 89%, 90%, 91%, 92%, 93%, 94%, 95%, 96%, 97%, 98%, 99% or 100% identical to the protospacer sequence. In a related embodiment, at least 10, 11, 12, 13, 14, 15, 16, 17, 18, 19, or 20 or more polynucleotides in the region of complementarity are identical to the protospacer motif. In a preferred embodiment, at least 10 of the 3′ most sequence in the protospacer motif is complementary to the crRNA sequence.

As used herein, the term “tracrRNA” refers to a trans-activating RNA that associates with the crRNA sequence through a region of partial complementarity and serves to recruit a Cas9 nuclease to the protospacer motif. In one embodiment, the tracrRNA is at least 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, or more nucleotides in length. In one embodiment, the tracrRNA is about 85 nucleotides in length.

In one embodiment, the crRNA and tracrRNA are engineered into one polynucleotide sequence referred to herein as a “single guide RNA” or “sgRNA.” The crRNA equivalent portion of the sgRNA is engineered to guide the Cas9 nuclease to target any desired protospacer motif. In one embodiment, the tracrRNA equivalent portion of the sgRNA is engineered to be at least 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, or more nucleotides in length.

The protospacer motif abuts a short protospacer adjacent motif (PAM), which plays a role in recruiting a Cas9/RNA complex. Cas9 polypeptides recognize PAM motifs specific to the Cas9 polypeptide. Accordingly, the CRISPR/Cas9 system can be used to target and cleave either or both strands of a double-stranded polynucleotide sequence flanked by particular 3′ PAM sequences specific to a particular Cas9 polypeptide. PAMs may be identified using bioinformatics or using experimental approaches. Esvelt et al., 2013, Nature Methods. 10(11):1116-1121, which is hereby incorporated by reference in its entirety.

As used herein, the terms “siRNA” or “short interfering RNA” refer to a short polynucleotide sequence that mediates a process of sequence-specific post-transcriptional gene silencing, translational inhibition, transcriptional inhibition, or epigenetic RNAi in animals (Zamore et al., 2000, Cell, 101, 25-33; Fire et al., 1998, Nature, 391, 806; Hamilton et al., 1999, Science, 286, 950-951; Lin et al., 1999, Nature, 402, 128-129; Sharp, 1999, Genes & Dev., 13, 139-141; and Strauss, 1999, Science, 286, 886). In certain embodiments, an siRNA comprises a first strand and a second strand that have the same number of nucleosides; however, the first and second strands are offset such that the two terminal nucleosides on the first and second strands are not paired with a residue on the complimentary strand. In certain instances, the two nucleosides that are not paired are thymidine resides. The siRNA should include a region of sufficient homology to the target gene, and be of sufficient length in terms of nucleotides, such that the siRNA, or a fragment thereof, can mediate down regulation of the target gene. Thus, an siRNA includes a region which is at least partially complementary to the target RNA. It is not necessary that there be perfect complementarity between the siRNA and the target, but the correspondence must be sufficient to enable the siRNA, or a cleavage product thereof, to direct sequence specific silencing, such as by RNAi cleavage of the target RNA. Complementarity, or degree of homology with the target strand, is most critical in the antisense strand. While perfect complementarity, particularly in the antisense strand, is often desired, some embodiments include one or more, but preferably 10, 8, 6, 5, 4, 3, 2, or fewer mismatches with respect to the target RNA. The mismatches are most tolerated in the terminal regions, and if present are preferably in a terminal region or regions, e.g., within 6, 5, 4, or 3 nucleotides of the 5′ and/or 3′ terminus. The sense strand need only be sufficiently complementary with the antisense strand to maintain the overall double-strand character of the molecule. Each strand of an siRNA can be equal to or less than 30, 25, 24, 23, 22, 21, or 20 nucleotides in length. The strand is preferably at least 19 nucleotides in length. For example, each strand can be between 21 and 25 nucleotides in length. Preferred siRNAs have a duplex region of 17, 18, 19, 29, 21, 22, 23, 24, or 25 nucleotide pairs, and one or more overhangs of 2-3 nucleotides, preferably one or two 3′ overhangs, of 2-3 nucleotides.

As used herein, the terms “miRNA” or “microRNA” s refer to small non-coding RNAs of 20-22 nucleotides, typically excised from ˜70 nucleotide foldback RNA precursor structures known as pre-miRNAs. miRNAs negatively regulate their targets in one of two ways depending on the degree of complementarity between the miRNA and the target. First, miRNAs that bind with perfect or nearly perfect complementarity to protein-coding mRNA sequences induce the RNA-mediated interference (RNAi) pathway. miRNAs that exert their regulatory effects by binding to imperfect complementary sites within the 3′ untranslated regions (UTRs) of their mRNA targets, repress target-gene expression post-transcriptionally, apparently at the level of translation, through a RISC complex that is similar to, or possibly identical with, the one that is used for the RNAi pathway. Consistent with translational control, miRNAs that use this mechanism reduce the protein levels of their target genes, but the mRNA levels of these genes are only minimally affected. miRNAs encompass both naturally occurring miRNAs as well as artificially designed miRNAs that can specifically target any mRNA sequence. For example, in one embodiment, the skilled artisan can design short hairpin RNA constructs expressed as human miRNA (e.g., miR-30 or miR-21) primary transcripts. This design adds a Drosha processing site to the hairpin construct and has been shown to greatly increase knockdown efficiency (Pusch et al., 2004). The hairpin stem consists of 22-nt of dsRNA (e.g., antisense has perfect complementarity to desired target) and a 15-19-nt loop from a human miR. Adding the miR loop and miR30 flanking sequences on either or both sides of the hairpin results in greater than 10-fold increase in Drosha and Dicer processing of the expressed hairpins when compared with conventional shRNA designs without microRNA. Increased Drosha and Dicer processing translates into greater siRNA/miRNA production and greater potency for expressed hairpins.

As used herein, the terms “shRNA” or “short hairpin RNA” refer to double-stranded structure that is formed by a single self-complementary RNA strand. shRNA constructs containing a nucleotide sequence identical to a portion, of either coding or non-coding sequence, of the target gene are preferred for inhibition. RNA sequences with insertions, deletions, and single point mutations relative to the target sequence have also been found to be effective for inhibition. Greater than 90% sequence identity, or even 100% sequence identity, between the inhibitory RNA and the portion of the target gene is preferred. In certain preferred embodiments, the length of the duplex-forming portion of an shRNA is at least 20, 21 or 22 nucleotides in length, e.g., corresponding in size to RNA products produced by Dicer-dependent cleavage. In certain embodiments, the shRNA construct is at least 25, 50, 100, 200, 300 or 400 bases in length. In certain embodiments, the shRNA construct is 400-800 bases in length. shRNA constructs are highly tolerant of variation in loop sequence and loop size.

As used herein, the term “ribozyme” refers to a catalytically active RNA molecule capable of site-specific cleavage of target mRNA. Several subtypes have been described, e.g., hammerhead and hairpin ribozymes. Ribozyme catalytic activity and stability can be improved by substituting deoxyribonucleotides for ribonucleotides at noncatalytic bases. While ribozymes that cleave mRNA at site-specific recognition sequences can be used to destroy particular mRNAs, the use of hammerhead ribozymes is preferred. Hammerhead ribozymes cleave mRNAs at locations dictated by flanking regions that form complementary base pairs with the target mRNA. The sole requirement is that the target mRNA has the following sequence of two bases: 5′-UG-3′. The construction and production of hammerhead ribozymes is well known in the art.

Polynucleotides can be prepared, manipulated and/or expressed using any of a variety of well established techniques known and available in the art. In order to express a desired polypeptide, a nucleotide sequence encoding the polypeptide, can be inserted into appropriate vector. Examples of vectors are plasmid, autonomously replicating sequences, and transposable elements. Additional exemplary vectors include, without limitation, plasmids, phagemids, cosmids, artificial chromosomes such as yeast artificial chromosome (YAC), bacterial artificial chromosome (BAC), or P1-derived artificial chromosome (PAC), bacteriophages such as lambda phage or M13 phage, and animal viruses. Examples of categories of animal viruses useful as vectors include, without limitation, retrovirus (including lentivirus), adenovirus, adeno-associated virus, herpesvirus (e.g., herpes simplex virus), poxvirus, baculovirus, papillomavirus, and papovavirus (e.g., SV40). Examples of expression vectors are pClneo vectors (Promega) for expression in mammalian cells; pLenti4/V5-DEST™, pLenti6/V5-DEST™, and pLenti6.2/V5-GW/lacZ (Invitrogen) for lentivirus-mediated gene transfer and expression in mammalian cells. In particular embodiments, the coding sequences of the chimeric proteins disclosed herein can be ligated into such expression vectors for the expression of the chimeric protein in mammalian cells.

“Expression control sequences,” “control elements,” or “regulatory sequences” present in an expression vector are those non-translated regions of the vector—origin of replication, selection cassettes, promoters, enhancers, translation initiation signals (Shine Dalgarno sequence or Kozak sequence) introns, a polyadenylation sequence, 5′ and 3′ untranslated regions—which interact with host cellular proteins to carry out transcription and translation. Such elements may vary in their strength and specificity. Depending on the vector system and host utilized, any number of suitable transcription and translation elements, including ubiquitous promoters and inducible promoters may be used.

The term “operably linked”, refers to a juxtaposition wherein the components described are in a relationship permitting them to function in their intended manner. In one embodiment, the term refers to a functional linkage between a nucleic acid expression control sequence (such as a promoter, and/or enhancer) and a second polynucleotide sequence, e.g., a polynucleotide-of-interest, wherein the expression control sequence directs transcription of the nucleic acid corresponding to the second sequence. In one embodiment, the terms “operably linked to an inhibitory RNA” and “operably linked to a polynucleotide used as a template for an inhibitory RNA” or equivalents are used interchangeably.

As used herein, “conditional expression” may refer to any type of conditional expression including, but not limited to, inducible expression; repressible expression; expression in cells or tissues having a particular physiological, biological, or disease state, etc. This definition is not intended to exclude cell type or tissue specific expression. Certain embodiments of the invention provide conditional expression of a polynucleotide-of-interest, e.g., expression is controlled by subjecting a cell, tissue, organism, etc., to a treatment or condition that causes the polynucleotide to be expressed or that causes an increase or decrease in expression of the polynucleotide encoded by the polynucleotide-of-interest.

Illustrative examples of inducible promoters/systems include, but are not limited to, steroid-inducible promoters such as promoters for genes encoding glucocorticoid or estrogen receptors (inducible by treatment with the corresponding hormone), metallothionein promoter (inducible by treatment with various heavy metals), MX-1 promoter (inducible by interferon), the “GeneSwitch” mifepristone-regulatable system (Sirin et al., 2003, Gene, 323:67), the cumate inducible gene switch (WO 2002/088346), tetracycline-dependent regulatory systems, etc.

Conditional expression can also be achieved by using a site specific DNA recombinase. According to certain embodiments of the invention, polynucleotides comprise at least one (typically two) site(s) for recombination mediated by a site specific recombinase. As used herein, the terms “recombinase” or “site specific recombinase” include excisive or integrative proteins, enzymes, co-factors or associated proteins that are involved in recombination reactions involving one or more recombination sites (e.g., two, three, four, five, six, seven, eight, nine, ten or more), which may be wild-type proteins (see Landy, Current Opinion in Biotechnology 3:699-707 (1993)), or mutants, derivatives (e.g., fusion proteins containing the recombination protein sequences or fragments thereof), fragments, and variants thereof. Illustrative examples of recombinases suitable for use in particular embodiments of the present invention include, but are not limited to: Cre, Int, IHF, Xis, Flp, Fis, Hin, Gin, ΦC31, Cin, Tn3 resolvase, TndX, XerC, XerD, TnpX, Hjc, Gin, SpCCE1, and ParA.

In particular embodiments, polynucleotides comprise a polyadenylation sequence 3′ of a polynucleotide encoding a polypeptide to be expressed. Polyadenylation sequences can promote mRNA stability by addition of a polyA tail to the 3′ end of the coding sequence and thus, contribute to increased translational efficiency. Recognized polyadenylation sites include an ideal polyA sequence (e.g., AATAAA, ATTAAA AGTAAA), an SV40 polyA sequence, a bovine growth hormone polyA sequence (BGHpA), a rabbit β-globin polyA sequence (rβgpA), or another suitable heterologous or endogenous polyA sequence known in the art.

In particular embodiments, the endonuclease is a Cas9 polypeptide obtained from the following illustrative list of bacterial species: Enterococcus faecium, Enterococcus italicus, Listeria innocua, Listeria monocytogenes, Listeria seeligeri, Listeria ivanovii, Streptococcus agalactiae, Streptococcus anginosus, Streptococcus bovis, Streptococcus dysgalactiae, Streptococcus equinus, Streptococcus gallolyticus, Streptococcus macacae, Streptococcus mutans, Streptococcus pseudoporcinus, Streptococcus pyogenes, Streptococcus thermophilus, Streptococcus gordonii, Streptococcus infantarius, Streptococcus macedonicus, Streptococcus mitis, Streptococcus pasteurianus, Streptococcus suis, Streptococcus vestibularis, Streptococcus sanguinis, Streptococcus downei, Neisseria bacilliformis, Neisseria cinerea, Neisseria flavescens, Neisseria lactamica, Neisseria meningitidis, Neisseria subflava, Lactobacillus brevis, Lactobacillus buchneri, Lactobacillus casei, Lactobacillus paracasei, Lactobacillus fermentum, Lactobacillus gasseri, Lactobacillus jensenii, Lactobacillus johnsonii, Lactobacillus rhamnosus, Lactobacillus ruminis, Lactobacillus salivarius, Lactobacillus sanfranciscensis, Corynebacterium accolens, Corynebacterium diphtheriae, Corynebacterium matruchotii, Campylobacter jejuni, Clostridium perfringens, Treponema vincentii, Treponema phagedenis, and Treponema denticola.

Cas9 polypeptides target double-stranded polynucleotide sequences flanked by particular 3′ PAM sequences specific to a particular Cas9 polypeptide. Each Cas9 nuclease domain cleaves one DNA strand. Cas9 polypeptides naturally contain domains homologous to both HNH and RuvC endonucleases. The HNH and RuvC-like domains are each responsible for cleaving one strand of the double-stranded DNA target sequence. The HNH domain of the Cas9 polypeptide cleaves the DNA strand complementary to the tracrRNA:crRNA or sgRNA. The RuvC-like domain of the Cas9 polypeptide cleaves the DNA strand that is not-complementary to the tracrRNA:crRNA or sgRNA.

In one embodiment, the endonuclease is a TALENs comprising one or more TALE domains. Transcription activator like effectors (TALEs) are natural type III effector proteins secreted by numerous species of Xanthomonas to modulate gene expression in host plants and to facilitate bacterial colonization and survival (Boch et al., Annu Rev Phytopathol 2010; Bogdanove et al., Curr Opin Plant Biol 2010). Recent studies of TALEs have revealed an elegant code linking the repetitive region of TALEs with their target DNA-binding site (Boch et al., Science 2009; Moscou et al., Science 2009). Common among the entire family of TALEs is a highly conserved and repetitive region within the middle of the protein, consisting of tandem repeats of mostly 33 or 34 amino acid segments. Repeat monomers differ from each other mainly in amino acid positions 12 and 13 (repeat variable di-residues), and recent computational and functional analyses have revealed a strong correlation between unique pairs of amino acids at positions 12 and 13 and the corresponding nucleotide in the TALE-binding site: NI to A, HD to C, NG to T, NN to G (and to a lesser degree A) (Boch et al., Science 2009; Moscou et al., Science 2009; Miller et al., Nat. Biotech 2011; Zhang et al., Nat. Biotech 2011).

In one embodiment, the endonuclease is homing endonuclease designed or engineered with one or more amino acid substitutions, additions, or deletions to enable the endonuclease to bind to a desired nucleic acid target sequence. Illustrative examples of homing endonucleases that may be engineered include, but are not limited to I-Onu I, I HjeMI, I-CpaMI, I-Sce I, I-Chu I, I-Dmo I, I-Cre I, I-Csm I, PI-Sce I, PI-T11 I, PI-Mtu I, I-Ceu I, I-Sce II, I-Sce III, HO, P1-Civ I, PI-Ctr I, PI-Aae I, PI-Bsu I, PI-Dha I, PI-Dra I, PI-May I, PI-Mch I, PI-Mfu I, PI-Mfl I, PI-Mga I, PI-Mgo I, PI-Min I, PI-Mka I, PI-Mle I, PI-Mma I, PI-Msh I, PI-Msm I, PI-Mth I, PI-Mtu I, PI-Mxe I, PI-Npu I, PI-Pfu I, PI-Rma I, PI-Spb I, PI-Ssp I, PI-Fac I, PI-Mja I, PI-Pho I, PI-Tag I, PI-Thy I, PI-Tko I, or PI-Tsp I.

In one embodiment, the endonuclease is a ZFN. ZFN comprise one or more zinc finger DNA binding domains and an endonuclease domain, e.g., Fok I. A number of methods are known in the art that can then be used to engineer one or more zinc finger proteins that have a high affinity for its target (e.g., preferably with a Kd of less than about 25 nM). A ZFP DNA binding domain can be designed or selected to bind to any suitable target site in the genetic locus with high affinity. WO 00/42219 comprehensively describes methods for design, construction, and expression of ATPs comprising zinc finger DNA binding domains for selected target sites. Each zinc finger recognizes approximately 3 bp of DNA. In one embodiment, zinc finger DNA binding domains can be designed to recognize 3, 6, 9, 12, 15, 18, 21, or 24 or more bp of DNA. Candidate zinc finger DNA binding domains for a given 3 bp DNA target sequence have been identified and modular assembly strategies have been devised for linking a plurality of the domains into a multifinger peptide targeted to the corresponding composite DNA target sequence. Other suitable method sknown in the art can also be used to design and construct nucleic acids encoding zinc finger DNA binding domains, e.g., phage display, random mutagenesis, combinatorial libraries, computer/rational design, affinity selection, PCR, cloning from cDNA or genomic libraries, synthetic construction and the like. (see, e.g., U.S. Pat. No. 5,786,538; Wu et al., PNAS 92:344-348 (1995); Jamieson et al., Biochemistry 33:5689-5695 (1994); Rebar & Pabo, Science 263:671-673 (1994); Choo & Klug, PNAS 91:11163-11167 (1994); Choo & Klug, PNAS 91: 11168-11172 (1994); Desjarlais & Berg, PNAS 90:2256-2260 (1993); Desjarlais & Berg, PNAS 89:7345-7349 (1992); Pomerantz et al., Science 267:93-96 (1995); Pomerantz et al., PNAS 92:9752-9756 (1995); Liu et al., PNAS 94:5525-5530 (1997); Griesman & Pabo, Science 275:657-661 (1997); Desjarlais & Berg, PNAS 91:11-99-11103 (1994)).

Illustrative retroviruses suitable for use in particular embodiments, include, but are not limited to: Moloney murine leukemia virus (M-MuLV), Moloney murine sarcoma virus (MoMSV), Harvey murine sarcoma virus (HaMuSV), murine mammary tumor virus (MuMTV), gibbon ape leukemia virus (GaLV), feline leukemia virus (FLV), spumavirus, Friend murine leukemia virus, Murine Stem Cell Virus (MSCV) and Rous Sarcoma Virus (RSV)) and lentivirus.

As used herein, the term “lentivirus” refers to a group (or genus) of complex retroviruses. Illustrative lentiviruses include, but are not limited to: HIV (human immunodeficiency virus; including HIV type 1, and HIV type 2); visna-maedi virus (VMV) virus; the caprine arthritis-encephalitis virus (CAEV); equine infectious anemia virus (EIAV); feline immunodeficiency virus (FIV); bovine immune deficiency virus (BIV); and simian immunodeficiency virus (SIV). In one embodiment, HIV based vector backbones (i.e., HIV cis-acting sequence elements) are preferred.

Retroviral vectors and more particularly lentiviral vectors may be used in practicing particular embodiments of the present invention. Accordingly, the term “retrovirus” or “retroviral vector”, as used herein is meant to include “lentivirus” and “lentiviral vectors” respectively.

As will be evident to one of skill in the art, the term “viral vector” is widely used to refer either to a nucleic acid molecule (e.g., a transfer plasmid) that includes virus-derived nucleic acid elements that typically facilitate transfer of the nucleic acid molecule or integration into the genome of a cell or to a viral particle that mediates nucleic acid transfer. Viral particles will typically include various viral components and sometimes also host cell components in addition to nucleic acid(s).

In various embodiments, vectors contemplated herein, comprise non-integrating or integration defective retrovirus. In one embodiment, an “integration defective” retrovirus or lentivirus refers to retrovirus or lentivirus having an integrase that lacks the capacity to integrate the viral genome into the genome of the host cells. In various embodiments, the integrase protein is mutated to specifically decrease its integrase activity. Integration-incompetent lentiviral vectors are obtained by modifying the pol gene encoding the integrase protein, resulting in a mutated pol gene encoding an integrative deficient integrase. Such integration-incompetent viral vectors have been described in patent application WO 2006/010834, which is herein incorporated by reference in its entirety.

Illustrative mutations in the HIV-1 pol gene suitable to reduce integrase activity include, but are not limited to: H12N, H12C, H16C, H16V, S81 R, D41A, K42A, H51A, Q53C, D55V, D64E, D64V, E69A, K71A, E85A, E87A, D116N, D1161, D116A, N120G, N1201, N120E, E152G, E152A, D35E, K156E, K156A, E157A, K159E, K159A, K160A, R166A, D167A, E170A, H171A, K173A, K186Q, K186T, K188T, E198A, R199c, R199T, R199A, D202A, K211A, Q214L, Q216L, Q221 L, W235F, W235E, K236S, K236A, K246A, G247W, D253A, R262A, R263A and K264H. 23. A vector comprising the polynucleotide of any one of claims 1-22.

Adeno-associated virus (AAV) is a small (.about.26 nm) replication-defective, nonenveloped virus, that depends on the presence of a second virus, such as adenovirus or herpes virus, for its growth in cells. AAV is not known to cause disease and induces a very mild immune response. AAV can infect both dividing and non-dividing cells and may incorporate its genome into that of the host cell. These features make AAV a very attractive candidate for creating viral vectors for gene therapy. In certain embodiments, a recombinant AAV (rAAV) is provided that comprises serotype that is effective in infecting cells of the nervous system. In some embodiments, the AAV comprises a serotype selected from the group consisting of: AAV9, AAV6, AAVrh10, AAV7M8, and AAV24YF.

“Recombinant AAV (rAAV) vectors” of the invention are typically composed of, at a minimum, a transgene and its regulatory sequences, and 5′ and 3′ AAV inverted terminal repeats (ITRs). It is this recombinant AAV vector which is packaged into a capsid protein and delivered to a selected target cell. In some embodiments, the transgene is a nucleic acid sequence, heterologous to the vector sequences, which encodes a polypeptide, protein, functional RNA molecule (e.g., miRNA, miRNA inhibitor) or other gene product, of interest. The nucleic acid coding sequence is operatively linked to regulatory components in a manner which permits transgene transcription, translation, and/or expression in a cell of a target tissue.

The AAV sequences of the vector typically comprise the cis-acting 5′ and 3′ inverted terminal repeat sequences (See, e.g., B. J. Carter, in “Handbook of Parvoviruses”, ed., P. Tijsser, CRC Press, pp. 155 168 (1990)). The ITR sequences are about 145 bp in length. Preferably, substantially the entire sequences encoding the ITRs are used in the molecule, although some degree of minor modification of these sequences is permissible. The ability to modify these ITR sequences is within the skill of the art. (See, e.g., texts such as Sambrook et al, “Molecular Cloning. A Laboratory Manual”, 2d ed., Cold Spring Harbor Laboratory, New York (1989); and K. Fisher et al., J Virol., 70:520 532 (1996)). An example of such a molecule employed in the present invention is a “cis-acting” plasmid containing the transgene, in which the selected transgene sequence and associated regulatory elements are flanked by the 5′ and 3′ AAV ITR sequences. The AAV ITR sequences may be obtained from any known AAV, including presently identified mammalian AAV types.

In one embodiment, an AAV comprising a Cas9 cDNA is packaged as a single-stranded AAV vector. In another embodiment, a dual AAV vector system is used in which the Cas9 cDNA is split into two halves and the two AAV vectors reconstitute the Cas9 gene by either splicing (trans-splicing), homologous recombination (overlapping), or a combination of the two (hybrid). In the dual AAV trans-splicing strategy, a splice donor (SD) signal is placed at the 3′ end of the 5′-half vector and a splice acceptor (SA) signal is placed at the 5′ end of the 3′-half vector. Upon co-infection of the same cell by the dual AAV vectors and inverted terminal repeat (ITR)-mediated head-to-tail concatemerization of the two halves, trans-splicing results in the production of a mature mRNA and full-size protein (Yan et al, 2000). Trans-splicing has been successfully used to express large genes in muscle and retina (Reich et al, 2003; Lai et al, 2005). Alternatively, the two halves of a large transgene expression cassette contained in dual AAV vectors may contain homologous overlapping sequences (at the 3′ end of the 5′-half vector and at the 5′ end of the 3′-half vector, dual AAV overlapping), which will mediate reconstitution of a single large genome by homologous recombination (Duan et al, 2001). T his strategy depends on the recombinogenic properties of the transgene overlapping sequences (Ghosh et al, 2006). A third dual AAV strategy (hybrid) is based on adding a highly recombinogenic region from an exogenous gene [i.e. alkaline phosphatase, AP (Ghosh et al, 2008, 2011)] to the trans-splicing vectors. The added region is placed downstream of the SD signal in the 5′-half vector and upstream of the SA signal in the 3′-half vector in order to increase recombination between the dual AAVs.

In one embodiment, the vector is an HSV based viral vector. The mature HSV virion consists of an enveloped icosahedral capsid with a viral genome consisting of a linear double-stranded DNA molecule that is 152 kb. In one embodiment, the HSV based viral vector is deficient in at least one essential HSV gene. Of course, the vector can alternatively or in addition be deleted for non-essential genes. In one embodiment, the HSV based viral vector that is deficient in at least one essential HSV gene is replication deficient. Most replication deficient HSV vectors contain a deletion to remove one or more intermediate-early, early, or late HSV genes to prevent replication. For example, the HSV vector may be deficient in an immediate early gene selected from the group consisting of: ICP4, ICP22, ICP27, ICP47, and a combination thereof. Advantages of the HSV vector are its ability to enter a latent stage that can result in long-term DNA expression and its large viral DNA genome that can accommodate exogenous DNA inserts of up to 25 kb. HSV-based vectors are described in, for example, U.S. Pat. Nos. 5,837,532, 5,846,782, and 5,804,413, and International Patent Applications WO 91/02788, WO 96/04394, WO 98/15637, and WO 99/06583, which are incorporated herein by reference. Preferably, the HSV vector is “multiply deficient,” meaning that the HSV vector is deficient in more than one gene function required for viral replication.

In one embodiment, the HSV vector comprises a serotype selected from the group consisting of: JΔNI5, JΔNI7, and JΔNI8.

The compositions disclosed herein can be formulated for delivery to a subject according to a variety of factors, e.g., the site and route of administration, that composition to be delivered, and the like.

### Formulations, Kits and Methods of Treatment

Therapeutic compositions comprising for use in targeting at least one CSF1-DAP12 pathway member are also provided. Such compositions typically comprise a CSF1-DAP12 pathway member modulator and a pharmaceutically acceptable carrier.

Such compositions can include a therapeutically effective amount of a CSF1-DAP12 pathway member modulator. As used herein, the term “therapeutically effective amount” or “effective amount” refers to an amount of a CSF1-DAP12 pathway member modulator that when administered alone or in combination with another therapeutic agent to a cell, tissue, or subject (e.g., a mammal such as a human or a non-human animal such as a primate, rodent, cow, horse, pig, sheep, etc.) is effective to prevent or ameliorate neuropathic pain in a subject in need of treatment, e.g., at risk or having neuropathic pain. A therapeutically effective dose further refers to that amount of the CSF1-DAP12 pathway member modulator sufficient to result in full or partial amelioration of symptoms of neuropathic pain. A therapeutically effective dose further refers to that amount of the CSF1-DAP12 pathway member modulator sufficient to provide for analgesia in a subject, e.g., local and/or systemic analgesia in subject in need of treatment. A therapeutically effective dose further refers to that amount of the CSF1-DAP12 pathway member modulator sufficient to provide for reduction of nerve injury induced mechanical hypersensitivity and microglia activation in the subject, e.g., as compared to prior to therapy with a CSF1-DAP12 pathway member modulator.

Various pharmaceutical compositions and techniques for their preparation and use are known to those of skill in the art in light of the present disclosure. For a detailed listing of suitable pharmacological compositions and techniques for their administration one may refer to the detailed teachings herein, which may be further supplemented by texts such as Remington's Pharmaceutical Sciences, 17th ed. 1985; Brunton et al., “Goodman and Gilman's The Pharmacological Basis of Therapeutics,” McGraw-Hill, 2005; University of the Sciences in Philadelphia (eds.), “Remington: The Science and Practice of Pharmacy,” Lippincott Williams & Wilkins, 2005; and University of the Sciences in Philadelphia (eds.), “Remington: The Principles of Pharmacy Practice,” Lippincott Williams & Wilkins, 2008.

The disclosed therapeutic compositions further include pharmaceutically acceptable materials, compositions or vehicle, such as a liquid or solid filler, diluent, excipient, solvent or encapsulating material, i.e., carriers. These carriers are involved in transporting the subject modulator from one organ, or region of the body, to another organ, or region of the body. Each carrier should be “acceptable” in the sense of being compatible with the other ingredients of the formulation and not injurious to the patient.

Another aspect of the present disclosure relates to kits for carrying out the administration of a CSF1-DAP12 pathway member modulator.

The present disclosure also provides methods of treating neuropathic pain in a subject, as well as formulation a CSF1-DAP12 pathway member modulator as disclosed herein for use in such a method. Such methods generally involve administering to a subject in need with a CSF1-DAP12 pathway member modulator as disclosed above, e.g., a polynucleotide comprising a neuron specific promoter (e.g., a trigeminal ganglion (TGG) or dorsal root ganglion (DRG) promoter) operably linked to a recombinant nucleic acid encoding an endonuclease that binds to a nucleotide sequence in a human colony stimulating factor 1 (hCSF1) gene or to a nucleotide sequence in another CSF1-DAP12 pathway member gene.

Subjects suitable for therapy include any subject having or at risk of neuropathic pain. Subjects include mammals, including both human and non-human mammals, e.g., primates, rodents, cows, horses, pigs, sheep, etc.

The methods of the present disclosure involve administration of a CSF1-DAP12 pathway member modulator in an amount effective to prevent or ameliorate neuropathic pain in a subject in need of treatment, e.g., at risk or having neuropathic pain. Such methods can provide full or partial amelioration of symptoms of neuropathic pain. Such methods can provide for analgesia in a subject in need of treatment, e.g., local and/or systemic analgesia. The present methods can provide for reduction of nerve injury induced mechanical hypersensitivity and microglia activation in the subject, e.g., as compared to prior to therapy with a CSF1-DAP12 pathway member modulator.

The CSF1-DAP12 pathway member modulators can be administered by any suitable route, e.g., by intrathecal bolus injection or infusion, intraganglionic injection, intraneural injection, subcutaneous injection, or intraventricular injection. For example, where a CSF1-DAP12 pathway member modulator is to be delivered to DRGs, the CSF1-DAP12 pathway member modulator can be administered to a subject by, for example, intrathecal bolus injection or infusion at multiple levels of the spinal column. CSF1-DAP12 pathway member modulators can be administered to the subject by intraganglionic injection directly into a single dorsal root ganglion, multiple dorsal root ganglia, or the trigeminal ganglion. For example, where the CSF1-DAP12 pathway member can be administered to the subject by intraneural injection into the nerve bundle (e.g. sciatic nerve, trigeminal nerve). In another example, the CSF1-DAP12 pathway member modulator is administered to the subject by subcutaneous injection at the peripheral nerve terminals (subdermal or internal organ wall). In another example, the CSF1-DAP12 pathway member modulator is administered to the subject by intraventricular injection (for trigeminal ganglion transduction).

For methods of treatment of central neuropathic pain, the CSF1-DAP12 pathway member modulators may be administered to a subject in need by delivery to the central nervous system, e.g., intraparenchymal administration, intracisternal administration, intracranial administration, intraspinal administration, stereotactic brain injection, and the like.

In general, subjects amenable to treatment according to the methods disclosed herein have or are at risk of neuropathic pain. In general, neuropathic pain is the result of an injury, disorder or malfunction affecting the peripheral or central nervous system. Neuropathic pain may result from disorders of the peripheral nervous system or the central nervous system (brain and spinal cord). Neuropathic pain may be divided into peripheral neuropathic pain, central neuropathic pain, or mixed (peripheral and central) neuropathic pain. For example, the pain can be triggered by an injury, but this injury may or may not involve actual damage to the nervous system. For example, nerves can be infiltrated or compressed by tumors, strangulated by scar tissue, or inflamed by infection. The pain frequently has burning, lancinating, or electric shock qualities. Persistent allodynia, pain resulting from a nonpainful stimulus such as a light touch, is also a common characteristic of neuropathic pain.

Examples of neuropathic pain include post herpetic (or post-shingles) neuralgia, reflex sympathetic dystrophy, components of cancer pain, phantom limb pain, entrapment neuropathy (e.g., carpal tunnel syndrome), and peripheral neuropathy (widespread nerve damage). Neuropathic pain can also be associated with diabetes, as well as chronic alcohol use, exposure to toxins (including many chemotherapeutic agents), and vitamin deficiencies.

Examples of conditions that can be associated with neuropathic pain include but are not limited to autoimmune disease, e.g. multiple sclerosis, metabolic diseases e.g. diabetic neuropathy (including peripheral, focal, proximal and autonomic), infection e.g. shingles, postherpetic neuralgia, vascular disease, trauma, pain resulting from chemotherapy, HIV infection/AIDS, spine or back surgery, post-amputation pain, central pain syndrome, postherpetic neuralgia, phantom limb, trigeminal neuralgia, reflex sympathetic dystrophy syndrome, nerve compression, stroke, spinal cord injury and cancer. Generally the lesion leading to pain can directly involve the nociceptive pathways Neuropathic pain can also be idiopathic

### EXPERIMENTAL

### Materials and Methods

Mouse Lines

All animal experiments were approved by the Institutional Animal Care and Use Committee at UCSF and were conducted in accordance with the NIH Guide for the Care and Use of Laboratory animals. Wild type BL6/C57 mice and CSF1R-EGFP mice were purchased from Jackson Laboratory. DAP12 ko, CSF1 fl/fl, Advillin-Cre, and Nestin-Cre mice were described previously. High (HA) and low autotomy (LA) rats were raised as previously described.

Surgeries and Intrathecal Injection

The spared nerve injury (SNI) model of neuropathic pain was used. In short, after anesthesia with 2% isoflurane, the sural and superficial peroneal branches of the sciatic nerve were tightly ligated with 8-0 silk sutures and then transected distal to the ligature, leaving the tibial nerve intact. The overlying muscle and skin were sutured, and the animals allowed to recover and then returned to their home cages. To analyze CSF1 transport from the DRG to the spinal cord, the L4 and L5 dorsal root were ligated with 8-0 silk sutures at the time of the peripheral nerve injury. Intrathecal injection was performed as previously described. Ten microliters of 3 ng/ml CSF1 (total of 30 ng) or 40 ng/ml CSF1 neutralizing antibody (total of 400 ng) were intrathecally injected. To study CSF1-induced microglia proliferation, CSF1 was injected daily for three days. To study CSF1-induced microglial gene induction, CSF1 was injected twice within 24 hours; spinal cord tissue was collected 24 hours after the first injection.

Antibodies

The following antibodies were used ATF3 (Santa Cruz, rabbit, 1:2000), BrdU (Abcam, rat, 1:400), CSF1 (R&D, goat, 1:1000), CSF1R (Millipore), CD11b (Abcam), NeuN (Millipore), BrdU (Abcam), GFP (Abcam, chicken, 1:2000), Ibal (Wako, rabbit, 1:1000), NPY (gift from J. Allen, rabbit, 1:5000), PKCy (Strategic Bio, guinea pig, 1:10,000). To detect primary antibodies, appropriate fluorophore-coupled secondary antibodies from Invitrogen (Alexa Fluor 488, 555, 594, 647) were used. To localize CSF1 in DRG neurons and in their processes, a bridge immunostaining protocol was used to detect the primary antiserum: anti-goat biotin IgG (Vector Laboratories, 1:500) and streptavidin coupled to an Alexa Fluor 488 or 594 (Invitrogen, 1:1000).

Immunohistochemistry

Mice were anesthetized with Avertin (250 mg/kg; 2, 2,2-Tribromoethanol, Sigma) and perfused transcardially with phosphate-buffered saline (PBS) followed by 10% formalin in PBS (Fisher Scientific) at room temperature (RT). Spinal cord and DRG were dissected, postfixed in the same fixative for 3 hours at RT, and then cryoprotected in 30% sucrose PBS overnight at 4° C. Fourteen mm (DRG) or 30 μm (spinal cord, coronal) cryostat sections were pre-incubated for 60 min at RT in PBS/0.3% Triton X-100 containing 10% normal goat serum (NGS) or normal horse serum (NHS) and then immunostained overnight at RT in PBS containing 0.3% Triton X-100, 1% NGS or NHS and the primary antibodies. After 3 washes in PBS, the sections were incubated for 1 hour with secondary antibodies, washed in PBS, mounted in Fluoromount-G (Southern Biotechnology) and coverslipped.

Microglia Proliferation

To monitor nerve injury- and CSF1-induced microglia proliferation, mice were injected with BrdU (100 mg/g body weight, i.p.) 2 hours prior to perfusion. Spinal cord sections were collected as described above. To immunolabel with anti-BrdU antibodies, tissue sections were treated with 1M HCl (10 min, on ice), 2M HCl (10 min, RT) and 2M HCl (20 min, 37° C.). Tissue sections were washed 5 times in PBS and then immunostained following the protocol described above.

Imaging and Image Analysis

Images were collected with a Carl Zeiss LSM 700 microscope. Image processing and quantification were performed with Fiji/ImageJ (NIH) and corresponding images (e.g. ipsilateral vs contralateral; CSF1 vs PBS; wt vs ko) were processed in an identical manner. To assess coexpression of CSF1 and ATF3, 14 μm sections from the L4 and L5 DRG ipsilateral and contralateral to the sciatic nerve injury were collected (6 mice/group). 6-8 sections were imaged per animal and the thresholding and particle analyzer function in Fiji/Image J were used to count neurons with a visible nucleus. The analysis was performed blind to group. To quantify Ibal immunoreactivity after intrathecal CSF1, PKCy immunostaining was used to define the ventral border of the superficial dorsal horn and thresholding was used to measure signal intensity. 3 mice/group and 3 images/mouse were analyzed. Images were processed automatically. Results are normalized to values obtained in mice that were injected with vehicle (PBS). BrdU immunoreactive cells in the dorsal horn were also counted automatically using thresholding and the particle analyzer (Fiji/ImageJ). To quantify BrdU expression over time after SNI, the superficial dorsal horn was outlined with NeuN and analyzed 4 mice/time point, 3 images/mouse ipsilateral to the injury. To quantify BrdU labeled cells in response to intrathecal vehicle (PBS) or CSF1, BrdU labeled cells in the gray matter dorsal to the central canal were counted (3-4 mice/group and at least 3 images/mouse).

For the quantification of signal intensities of CSF1R, CSF1R-GFP and Ibal in dorsal horn microglia, 30 μm cryosections of the lumbar enlargement from 3-4 mice per group were collected. Confocal images were taken from the 3 sections showing the highest microglia signals in each animal. The border of the dorsal horn was outlined, all microglia cells were identified using an independent microglia marker (Ibal or CD11b), and signal intensities within this mask were analyzed using Fiji/Image J.

RNA-Seq

Ipsilateral and contralateral DRGs and the dorsal quadrant of the spinal cords were collected 7d after nerve injury. RNA was purified with QIAgen RNeasy Mini Kit with DNase I digestion. RNA-Seq libraries were built with Epicentre ScriptSeq mRNA-Seq Library Preparation Kit and were sequenced by Illumina HiSeq 2000. Differential expression testing was performed using Cuffdiff 1.3.0 using default parameters. Resulting significant gene lists were filtered for genes with an absolute fold change greater than 2.

Quantitative RT-PCR

Mice were anesthetized with Avertin and perfused transcardially with PBS. In mice with a peripheral nerve injury, the investigators collected L4-6 DRGs and dorsal spinal cord ipsilateral and contralateral to the injury. For the mice that received an intrathecal CSF1 injection, the investigators collected the entire lumbar spinal cord. Total RNA was purified with Trizol-chloroform (Ambion) and treated with DNase (Ambion). cDNA was synthesized with SuperScript III First Strand Supermix or First-Strand Synthesis System (Invitrogen). Quantitative RT-PCR was performed using Bio-Rad CFX Connect and Maxima SYBR Green/ROX qPCR Mastermix (Thermo Scientific). All primers described below (TABLE 1) were designed using the NCBI Primer-BLAST program. B-actin was used as the internal control for all the DRG samples, and Snap25 was used as the internal control for all spinal cord samples. In addition to above protocol, qRT-PCR was performed as previously described (Braz et al., Neuron. 2012, 74:663-675).

TABLE 1

| | Forward | Reverse |
|---|---|---|
| Mouse | TGCTAAGTGCTCTAGCCGAG | CCCCCAACAGTCAGCAAGAC |
| CSF1 | (SEQ ID NO: 1) | (SEQ ID NO: 2) |
| Mouse | CCACACCCGCCACCAGTTCG | TACAGCCCGGGGAGCATCGT |
| β-actin | (SEQ ID NO: 3) | (SEQ ID NO: 4) |
| Mouse | ACGTACAGCGGAGCCTCAT | CATGACCCGGAAGCAGTTGT |
| IL34 | (SEQ ID NO: 5) | (SEQ ID NO: 6) |
| Mouse | CCGAGGTCAAGGGACAGCGGA | TGCCTCTGTGTGTTGAGGTCACTGT |
| DAP12/ | (SEQ ID NO: 7) | (SEQ ID NO: 8) |
| Tyrobp | | |
| Mouse | GAGTCTGCCTCCGTGTCCGC | TACGTGAGCGGCCAGGGTCT |
| CD11b/ | (SEQ ID NO: 9) | (SEQ ID NO: 10) |
| Itgam | | |
| Mouse | GCCTCTGGTGGAGTCTGCGTG | CGCCCAAATAACAGGCCTCAGCA |
| CX3CR1 | (SEQ ID NO: 11) | (SEQ ID NO: 12) |
| Mouse | CAGGTTCGAGAGGTCTGACG | AAGTGTACAAGTCCGCGTCC |
| BDNF | (SEQ ID NO: 13) | (SEQ ID NO: 14) |
| Mouse | GGGGGCATAGAGGCAGACGCT | GGGCATCCTCGTCACCAAACGG |
| CatS | (SEQ ID NO: 15) | (SEQ ID NO: 16) |
| Mouse | CGACTATGTGGTCCCAGCTC | GCGTCTGAATCGCAAATGCT |
| P2X4 | (SEQ ID NO: 17) | (SEQ ID NO: 18) |
| Mouse | GGGCAGCGTGGGAACC | GCTTCCAGGGGATACGGAAC |
| Irf8 | (SEQ ID NO: 19) | (SEQ ID NO: 20) |
| Mouse | TGGGGACAACACCATCTTCA | CTGGAAGTCACGGCTTTTGT |
| Irf5 | (SEQ ID NO: 21) | (SEQ ID NO: 22) |
| Rat | AAACAGCACATGGCTGAGAC | GCATAGGGTGGGTTCATCTGT |
| DAP12/ | (SEQ ID NO: 23) | (SEQ ID NO: 24) |
| Tyrobp | | |
| Rat | ACCACTGACTTGCTGGCCCCG | CGACGGGTGCTTTCCAGGGAC |
| Snap25 | (SEQ ID NO: 25) | (SEQ ID NO: 26) |
| Csf1r | ACACGCACGGCCACCATGAA | GCATGGACCGTGAGGATGAGGC |
| | (SEQ ID NO: 27) | (SEQ ID NO: 28) |
| Trem1 | ACTGCTGTGCGTGTTCTTTG | GCCTTCTGGCTGTTGGCATA |
| | (SEQ ID NO: 29) | (SEQ ID NO: 30) |
| Trem3 | CAAGATGTGGGGCTGTACCA | AAGCCACACGTCAGAACGAT |
| | (SEQ ID NO: 31) | (SEQ ID NO: 32) |
| Snap25 | AGCGGACAGCATCCTCCGGAG | GTCTGCGTCTTCGGCCATGGT |
| | (SEQ ID NO: 33) | (SEQ ID NO: 34) |

In Situ Hybridization

In situ hybridization (ISH) was performed using the Panomics' QuantiGene ViewRNA tissue assay (Affymetrix/Panomics), with a probe set designed for the three variants of the mouse Csf1 coding sequence (NM_007778.4, NM_001113530.1, and NM_001113529.1). The signal was detected using an alkaline phosphatase reaction with a fluorescent Fast Red substrate. The following protocol was used to combine ISH with immunohistochemistry for ATF3. The mice were deeply anesthetized and transcardially perfused with 10% formalin as above. Twelve μm cryostat sections collected on glass slides were immersed in 10% formalin for 10 minutes and then processed according to the manufacturer's ISH protocol. Protease treatment for 12 minutes was optimal for combining ISH with immunohistochemistry. Following the ISH steps, the slides were blocked in 5% normal goat serum/0.1M PBS (without Triton X-100) for one hour at RT and then processed for immunostaining as above.

Behavior Analysis

All behavioral assays were performed as previously described33 in a blinded manner. Motor coordination was tested on an accelerating rotarod (Ugo Basile, Model #7650). The duration that the mouse spent on the rotarod was recorded, with a cutoff of 300 sec. Prior to testing, each mouse received three training trials. For the Hargreaves' plantar test of heat pain sensitivity, mice were placed in clear plastic chambers on a glass surface through which a radiant heat source was focused on the hindpaw. In the hot plate test, the latency to lick or flinch the hindpaws, or to jump, was recorded. The responses were monitored at three different temperatures (48° C., 52.5° C., 55° C.). To test mechanical responsiveness, mice were placed into clear plastic chambers on a wire mesh grid and the hindpaw was stimulated with graded von Frey filaments. Withdrawal thresholds were determined using the up-down method34.

Statistical Analysis

Data are presented as mean±standard error (SEM). Student's t test and two-way repeatedmeasures ANOVA (Tukey's post hoc test) were used to analyze gene expression changes, immunofluorescence intensities, cell counts and behavioral results. Statistical significance: * p≤0.05, ** p≤0.01, *** p≤0.001, **** p≤0.0001.

### EXAMPLES

### Example 1: Peripheral Nerve Injury Induces hCSF1

The present inventors have discovered that peripheral nerve injury in mice induces de novo expression of the cytokine, (macrophage) colony stimulating factor 1 (CSF1), in injured DRG neurons. The CSF1 is transported to the spinal cord where it targets the microglial CSF1 receptor (CSF1R). Cre-mediated deletion of Csf1 from sensory neurons completely prevented the hypersensitivity and significantly reduced the microglia activation produced by nerve injury. In contrast, intrathecal (spinal) injection of CSF1 not only activates microglia, but also induces mechanical hypersensitivity comparable to that produced by nerve injury. Downstream of the microglial CSF1R, it was found that both nerve injury and intrathecal CSF1 upregulate DAP12, an adaptor protein that is central to microglial signaling.

DAP12 deletion abrogates both nerve injury and CSF1-induced mechanical hypersensitivity. DAP12 is also required for the nerve injury and CSF1-induced early upregulation of brain-derived neurotrophic factor (BDNF) and cathepsin S, microglial genes implicated in the development of neuropathic pain, but not for nerve injury or CSF1-induced microglial proliferation. The results disclosed herein demonstrate that CSF1 is an important signal between injured sensory neurons and a microglial, DAP12-dependent induction of genes required for the development of nerve injury-induced neuropathic pain.

The cytokine CSF1, plays a role in the differentiation and maintenance of the myeloid lineage population, including microglia, and the CSF1 receptor, CSF1R, is also required for microglia development. Moreover, in the adult CNS, CSF1R is only expressed in microglia.

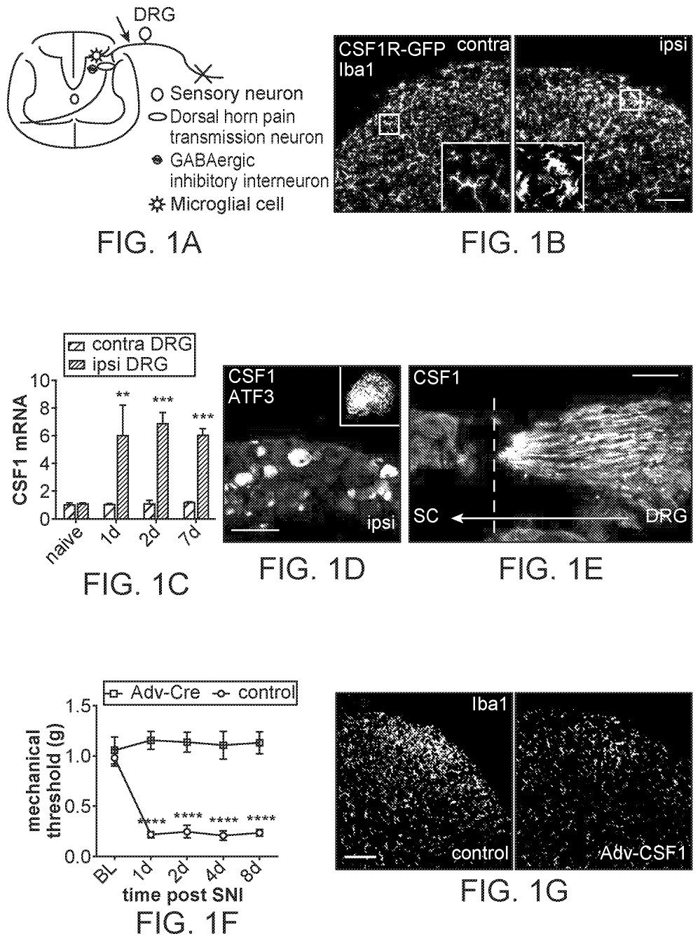

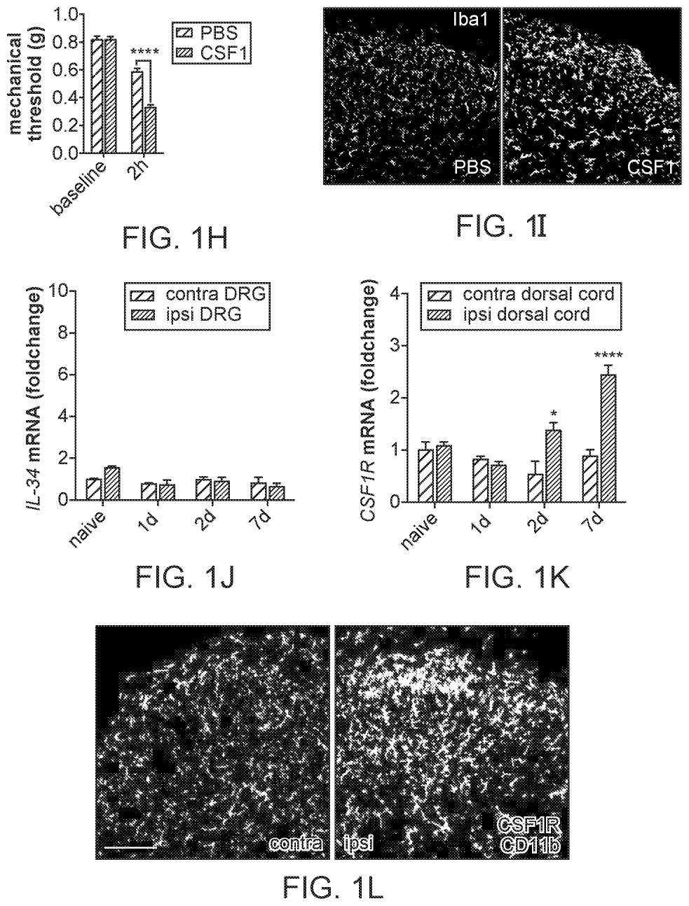

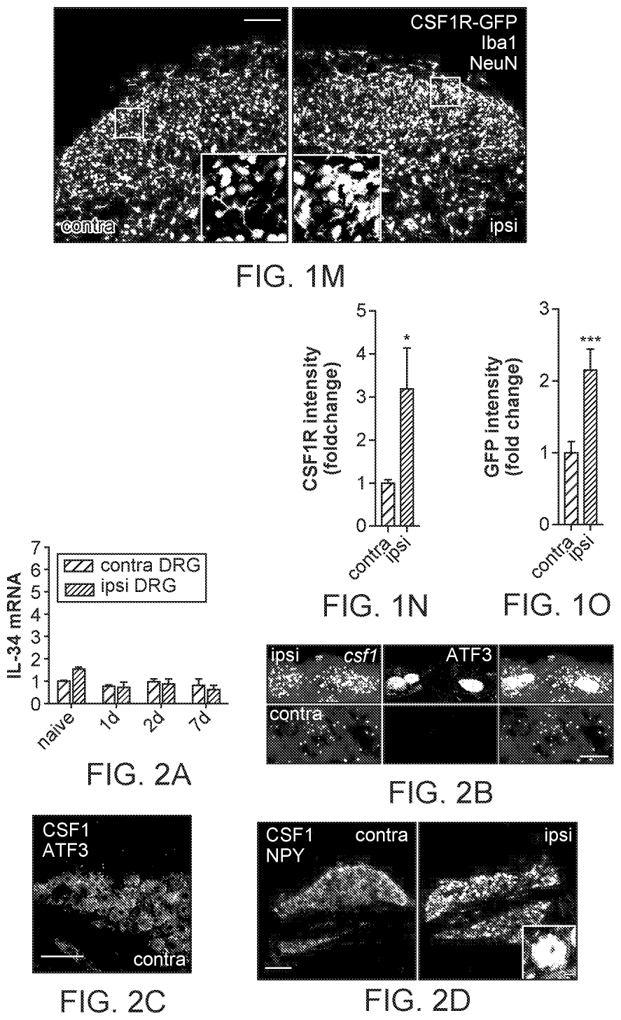

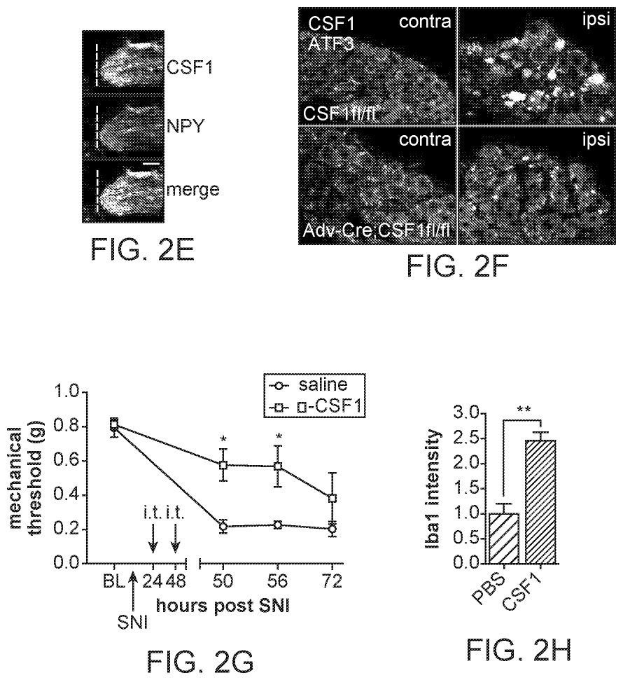

A partial sciatic nerve injury (SNI) model of neuropathic pain was used to monitor the behavioral changes as well as the molecular consequences of injury in sensory neurons of the DRG and in the lumbar spinal cord (FIG. 1A). First, by monitoring Ibal expression in a CSF1R-GFP reporter, it was shown that CSF1R is indeed exclusively expressed in microglia in the spinal cord and, as for Ibal, is upregulated in activated microglia after SNI (FIG. 1B). In addition, within one day of SNI a significant induction of Csf1 mRNA in the L4-L6 DRG ipsilateral to the nerve injury (FIG. 1C) was recorded. No DRG induction of a second CSF1R ligand, IL-34, which is also required for microglial development, was observed (FIG. 2A).

FIG. 1: (FIG. 1A) Schematic illustrating key neuroanatomical structures: sciatic nerve afferent fibers; sensory neurons in DRG; GABAergic inhibitory interneurons and microglia that regulate dorsal horn pain transmission (PT) neurons; X: sciatic nerve injury; arrow: dorsal root ligature site. (FIG. 1B) Increased dorsal spinal cord Ibal and GFP labeling in CSF1R-GFP reporter mouse ipsilateral to SNI. Inset: morphology of resting (left; control) and activated (right; injured) microglia. (FIG. 1C) Rapid induction of Csf1 mRNA in DRG after SNI. (FIG. 1D) Co-expression of ATF3 and CSF1 in DRG neurons ipsilateral to injury (1 day). (FIG. 1E) Accumulation of CSF1 at the dorsal root ligature. (FIG. 1F) Advillin-Cre mediated Csf1 deletion from sensory neurons prevents the development of SNI-induced mechanical hypersensitivity (n=5-6 mice/group). (FIG. 1G) Csf1 deletion from sensory neurons greatly reduces microglia activation ipsilateral to the SNI. (FIG. 1H) Intrathecal CSF1 produces a mechanical hypersensitivity significantly greater than that induced by the PBS vehicle (n=7 mice/group). (FIG. 1I) Intrathecal CSF1 also activates dorsal horn microglia. Scale bar: 100 μm (FIG. 1B, D, H, I); 200 μm (FIG. 1E). Mean±SEM, Two-way ANOVA, Tukey's posthoc analysis, * p≤0.05, ** p≤0.01, *** p≤0.001, **** p≤0.0001. (FIG. 1J) qRT-PCR shows that there is no induction of IL-34; (FIG. 1K) qRT-PCR illustrates Csf1 r induction in the dorsal cord ipsilateral to the nerve injury compared to the contralateral side. N=3 mice/time point. (FIG. 1L) CSF1R (immunostaining) co-localizes with the microglial marker CD11b and both markers are induced in the dorsal horn after nerve injury (3d post injury). Scale bar equals 100 μm; (FIG. 1M) At 3 days after nerve injury there is complete CSF1R-GFP co-localization with the microglial marker Ibal, and none with the neuronal marker, NeuN. White square shows enlarged region. Scale bar equals 100 μm; (FIG. 1N) Quantification of CSF1R immunostaining in CD11b positive cells in the superficial dorsal horn 3 days after nerve injury; (FIG. 1O) Quantification of GFP intensity in Ibal positive cells in the superficial dorsal horn 3 days after nerve injury. N=3-4 mice/group.

FIG. 2: (FIG. 2A) There is no induction of IL34 mRNA in DRG after SNI (n=3 mice/group, ipsilateral versus contralateral: Mean±SEM, 2-Way ANOVA, Tukey's multiple comparison test, not significant). (FIG. 2B) Combined in situ hybridization and immunocytochemistry illustrates de novo expression of Csf1 mRNA in injured DRG neurons that co-express ATF3. Scale bar: 10 μm. (FIG. 2C) Neither CSF1 nor ATF3 protein are expressed in DRG neurons contralateral to the nerve injury (4d post injury). (FIG. 2D) De novo expression of CSF1 protein in injured DRG neurons ipsilateral to the nerve injury colocalizes with NPY (Inset), a neuropeptide that is also only expressed in neurons after nerve injury. Scale bar: 200 μm and 10 μm (Inset) (FIG. 2E) Concurrent L4 and L5 dorsal root ligation and SNI results in the accumulation of CSF1 and NPY at the ligature. Co-localization of CSF1 with NPY establishes that the CSF1 transport is intra-axonal. Dashed line denotes ligatures. Scale bar: 200 μm. (FIG. 2F) ATF3 expression persists in DRG neurons from Adv-Cre; CSf1 fl/fl mice after nerve injury, despite complete loss of CSF1 induction. Upper panels: CSF1 fl/fl control mice; Lower panels: Advillin-Cre; CSF1 fl/fl mice. (FIG. 2G) Intrathecal CSF1 neutralizing antibody (24 and 48 h post SNI) reduces nerve injury-induced mechanical hypersensitivity (n=6 mice/group) (FIG. 2H) Quantification of Ibal signal intensities in the dorsal horn (FIG. 1H) shows significant increase of Ibal after intrathecal CSF1 (30 ng daily for 3 days) compared to vehicle (PBS). Values are normalized to immunostaining observed after PBS; n=3 mice/group, unpaired t-test, ** p≤0.01.

### Example 2: CSF1 is De Novo Induced in Injured Sensory Neurons and Transported to the Spinal Cord, where it Engages CSF1 Receptor (CSF1R)-Expressing Microglia

To identify the genes that are upregulated in DRG and dorsal horn after nerve injury and the signals through which injured sensory neurons interact with microglia to produce pain, an RNA-Seq analysis was first performed after nerve injury (FIG. 1A). Although many studies have reported transcriptional changes after nerve injury, few examined both DRG and spinal cord and most were performed using microarray (LaCroix-Fralish et al., Pain. 2011, 152:1888-1898; Perkins et al., Mol. Pain. 2014, 10:7). A dramatic upregulation of colony-stimulating factor 1 (Csf1) in the ipsilateral DRG and of its receptor (Csf1 r) was found in the ipsilateral dorsal cord after nerve injury (TABLE 2).

This finding is particularly important as CSF1 is an essential factor added to culture medium to expand microglia in vitro (Suzumura et al., J. Neuroimmunol. 1990, 30:111-120; Smith et al., J. Neuroinflammation. 2013, 10:85), and CSF1R is required in vivo for microglia development (Elmore et al., Neuron. 2014, 82:380-397). In fact, Csf1 r is among the earliest genes expressed in microglia progenitors in yolk sac during microglia development (Ginhoux et al., Science. 2010, 330:841-845; Schulz et al., Science. 2012, 336:86-90). The expression of IL-34, another CSF1R ligand (Wang et al., Nat. Immunol. 2012, 13:753-760), did not change (TABLE 2). qRT-PCR confirmed the finding that Csf1, but not 11-34, is induced in the DRG (FIG. 1C; FIG. 1J), and that Csf1 r is induced in the dorsal spinal cord (FIG. 1K) after nerve injury.

TABLE 2
RNA-Seq analysis of DRG and dorsal cord after peripheral nerve injury.
Relative expression levels (Fragments Per Kilobase of exon per
Million mapped fragments: FPKM) for selected genes
in the DRG and dorsal spinal cord 7d after nerve injury.

| Gene | Tissue | Contralateral | Ipsilateral | Fold increase |
|---|---|---|---|---|
| Csf1 | DRG | 11.02 | 63.14 | 63.14 |
| Il-34^{a} | DRG | 10.92 | 14.43 | 1.32 |
| CcI21a^{a} | DRG | 1.66 | 1.85 | 1.11 |
| CcI21b^{a} | DRG | 4.7 | 3.28 | 0.7 |
| CcI21c^{a} | DRG | 0 | 0 | — |
| Csf1r^{b} | Dorsal Cord | 16.72 | 60.97 | 3.65 |
| Tyrobp | Dorsal Cord | 9.29 | 38.03 | 4.09 |
| Cx3cr1^{b} | Dorsal Cord | 12.65 | 36.61 | 2.89 |
| Trem1^{c} | Dorsal Cord | 0 | 0 | — |
| Trem3^{c} | Dorsal Cord | 0 | 0.07 | — |
| Ccr7^{d} | Dorsal Cord | 0 | 0 | — |
| Cxcr3^{d} | Dorsal Cord | 0 | 0 | — |
| Cd200^{e} | DRG | 40.76 | 56.92 | 1.40 |
| Cd22^{e} | DRG | 0.08 | 0.11 | 1.38 |
| Cd47^{e} | DRG | 43.72 | 42.53 | 0.97 |
| Hspd1^{e} | DRG | 166.02 | 155.70 | 0.94 |
| Icam5^{e} | DRG | 0.27 | 0.40 | 1.48 |

^{a}Genes not upregulated in DRG after nerve injury;
^{b}Genes predominately expressed in microglia;
^{c}Genes exclusively expressed in monocytes;
^{d}CCL21 receptors;
^{e}Genes encoding neuronal membrane proteins that reportedly counteract microglia activation

Combined in situ hybridization for Csf1 mRNA and immunohistochemical localization of ATF3, a marker of cells with damaged peripheral axons, showed that the induction of Csf1 is limited to injured DRG neurons (FIG. 2B). Double immunostaining showed that all CSF1+ neurons co-expressed ATF3 and most ATF3+ neurons co-expressed CSF1 (FIG. 1D; FIG. 2C). The de novo CSF1 expression occurred in small diameter nociceptive and non-nociceptive, large diameter neurons (FIG. 1D; FIG. 2D). In fact, co-expression of CSF1 with ATF3 was observed as early as 12 hours after nerve injury, also in a mixed population of neurons. As CSF1 could not be detected in DRG neurons in the absence of injury (FIG. 1D), it was concluded that nerve injury induces de novo CSF1 expression in the injured sensory neurons.

To address CSF1 trafficking after its induction in the SNI model, the L4-L6 dorsal roots (between the DRG and spinal cord; FIG. 1A, arrow) were concurrently ligated and demonstrated damming of CSF1 at the ligature (FIG. 1E). This result confirmed that the CSF1 transport to the spinal cord was intra-axonal. FIG. 2E illustrates double labeling for CSF1 and neuropeptide Y, which is also expressed in DRG neurons only after peripheral nerve injury. Co-expression in DRG neurons and at the ligature site of CSF1 and NPY (FIG. 2D-2E), a peptide that is upregulated in injured sensory neurons (Hokfelt et al., Peptides. 2007, 28:365-372), confirmed the intra-axonal transport of CSF1. Next, using a CSF1R-GFP reporter mouse (Burnett et al., J. Leukoc. Biol. 2004, 75:612-623), and by immunostaining for CSF1R, CSF1R was found to be expressed exclusively in spinal cord microglia and is indeed upregulated after nerve injury (FIG. 1B; FIG. 1L-1O). A corresponding CSF1 increase in the dorsal horn was not observed, which suggests that the CSF1 is rapidly released after its transport to the cord.

### Example 3: CSF1 is Necessary and Sufficient for Nerve Injury-Induced Microglia Activation in the Spinal Dorsal Horn

The functional impact of CSF1 was addressed by selectively deleting Csf1 from DRG neurons by crossing a floxed Csf1 mouse with another (Advillin-Cre) in which Cre-recombinase is expressed only in sensory neurons (FIG. 2F). The ATF3 response of injured sensory neurons was not altered in these mice (FIG. 2F). However, Csf1 deletion prevented the hypersensitivity produced by nerve injury (FIG. 1F) and greatly reduced microglia activation (FIG. 1G). Similarly, intrathecal injection of a CSF1 neutralizing antibody significantly reduced the hypersensitivity produced by nerve injury (FIG. 2G). In contrast, intrathecal injection of CSF1 not only provoked a significant (within 2 h) mechanical hypersensitivity comparable to that produced by nerve injury (FIG. 1H), but also activated spinal cord microglia (FIG. 1I, FIG. 2H) to a significantly greater extent than did the vehicle (PBS). Thus, these results show that CSF1 is both a necessary and sufficient contributor to nerve injury-induced mechanical hypersensitivity and microglia activation.

### Example 4: DAP12 Mediates Nerve Injury- and CSF1-Induced Microglial Gene Upregulation and Pain

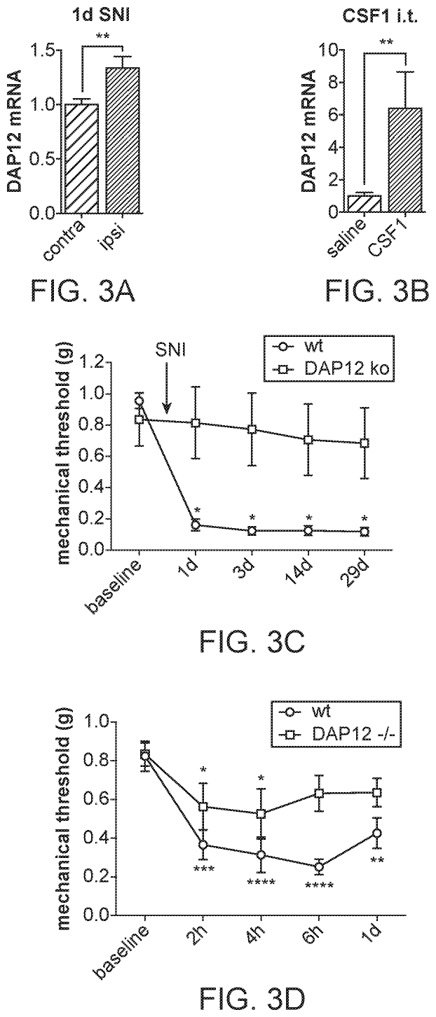

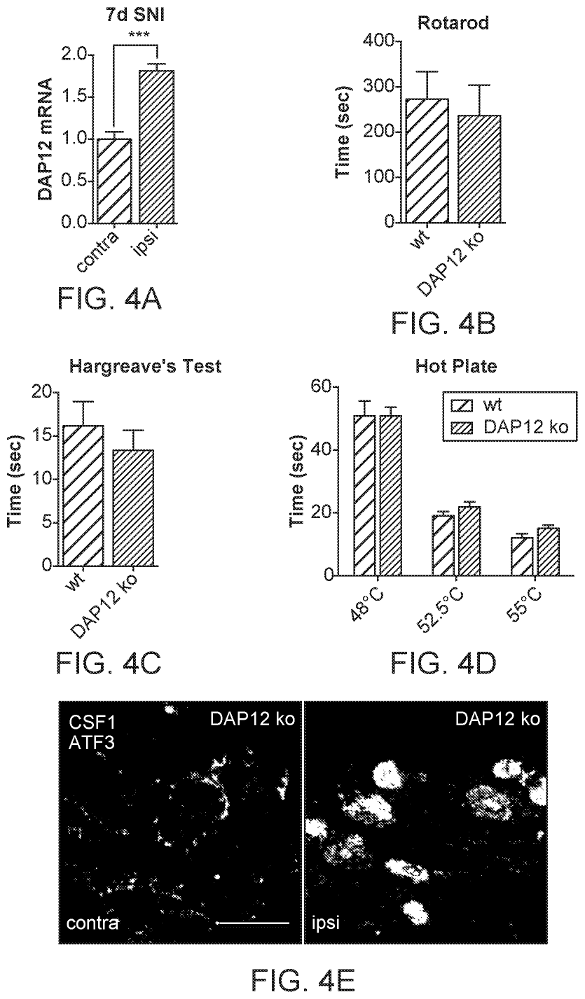

The signal transduction pathway downstream of CSF1 and CSF1R was studied by looking at the membrane adaptor protein DAP12, which is central to microglial functionality. FIG. 3A shows that peripheral nerve injury increased the level of DAP12 mRNA in the dorsal spinal cord. The increase was significant within 1 day of injury and lasted for at least 7 days (FIG. 4A). Intrathecal CSF1 also induced DAP12 expression (FIG. 3B). Deletion of DAP12 prevented both nerve injury- and intrathecal CSF1-induced mechanical hypersensitivity (FIG. 3C-3D). As DAP12 ko mice have normal baseline pain and motor behavior (FIG. 4B-4D), the failure to develop hypersensitivity did not result from a general pain processing or motor deficit. Furthermore, DAP12 deletion did not influence the de novo expression of CSF1 in DRG neurons (FIG. 4E). Taken together, these results demonstrate that the induction of CSF1 in sensory neurons is required for the development of neuropathic pain and that its contribution is dependent upon downstream DAP12 signaling in microglia.

FIG. 3: (FIG. 3A) Upregulation of DAP12 mRNA (qPCR) in dorsal spinal cord ipsilateral to SNI (1 day). (FIG. 3B) Upregulation of DAP12 mRNA (qPCR) in dorsal spinal cord (bilateral) 1 day after intrathecal CSF1. (FIG. 3C) DAP12 ko mice do not develop mechanical hypersensitivity after SNI (n=5-6 mice/group). (FIG. 3D) DAP12 ko mice do not develop mechanical hypersensitivity after intrathecal CSF1 (n=7 mice/group). The mild hypersensitivity observed in the DAP12 ko mice is comparable to that produced by PBS in wt mice in FIG. 1, Panel h. Student's t-test or 2-way ANOVA, Tukey's posthoc analysis, p≤0.05, ** p≤0.01, *** p≤0.001, **** p≤0.0001

FIG. 4: (FIG. 4A) DAP12 mRNA upregulation in ipsilateral dorsal cord persists 7 days after SNI (n=3 mice/group). (FIG. 4B) Motor performance in the rotorod test is normal in DAP12 ko mice (n=7 mice/group, no difference compared to wt). (FIG. 4C) DAP12 ko mice have normal responses to noxious heat (Hargreaves' test; n=6-7 mice/group; no difference compared to wt). (FIG. 4D) DAP12 ko mice have normal responses to noxious heat in the hot plate test, at several temperatures (n=6-7 mice/group; no difference compared to wt). (FIG. 4E) de novo expression of CSF1 in injured (ATF3-immunoreactive) DRG neurons after SNI persists in DAP12 ko mice. Scale bar=50 μm. Student's t-test, *** p≤0.001

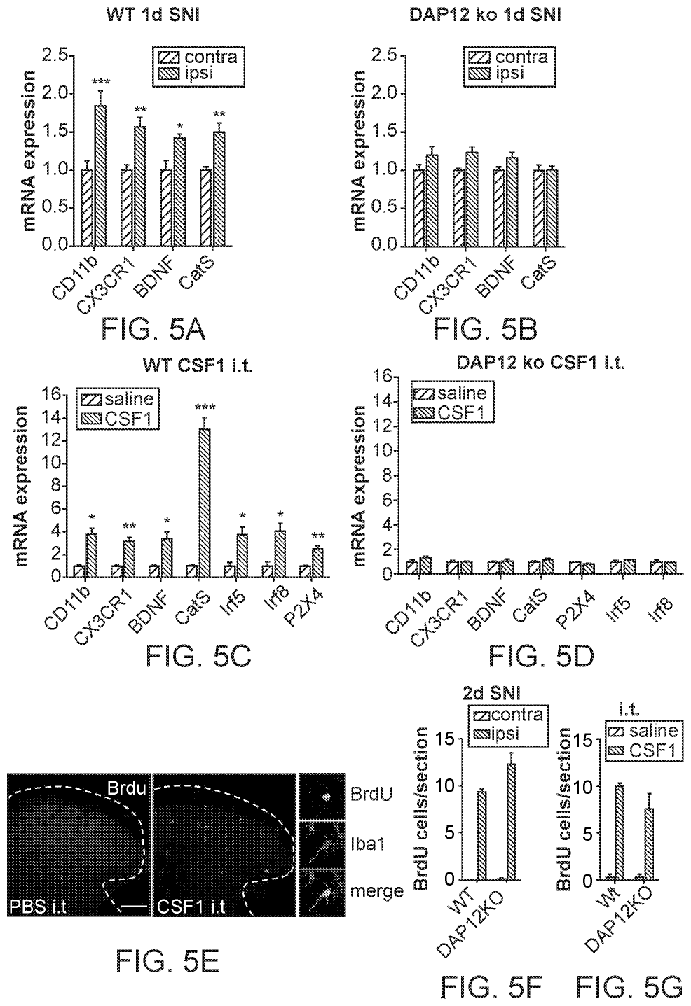

The changes in spinal cord gene expression after nerve injury were monitored and their dependence on DAP12 was assessed. Early time points were analyzed as genes induced shortly after nerve injury are more relevant to initiation of the neuropathic pain condition. One day post injury, a significant increase of the microglial specific genes that encode CD11b and CX3CR1, as well as BDNF and cathepsin S (FIG. 5A) was recorded, both of which are implicated in neuropathic pain. As there is no microglia proliferation one day after nerve injury, the microglial gene induction observed at this time point must derive from resident, rather than proliferating microglia. Intrathecal CSF1 at the same time point recapitulated this pattern of gene upregulation (FIG. 5C).

Both nerve injury and CSF1-induced gene upregulation were completely DAP12-dependent (FIG. 5B; FIG. 5D). Intrathecal CSF1 also induced upregulation of the P2X4 subtype of purinergic receptor as well as Irf8 and Irf5, which encode transcription factors that regulate BDNF and cathepsin S expression, again in a completely DAP12-dependent manner (FIG. 5C-5D). These results demonstrate that nerve injury-induced upregulation of microglial genes considered essential to sensitizing spinal cord pain transmission circuitry and to generating neuropathic pain involves a CSF1-CSF1R-DAP12-dependent microglial signaling pathway.

FIG. 5: (FIG. 5A) Upregulation of several microglial genes in the dorsal spinal cord 1 day after SNI (n=4-8 mice/group). (FIG. 5B) DAP12 ko prevents nerve injury-induced gene induction (n=4-5 mice/group). (FIG. 5C) Intrathecal CSF1 in wt mice induces microglial genes (n=3-4 mice/group). (FIG. 5D) DAP12 ko prevents intrathecal CSF1-induced microglial gene upregulation (n=4 mice/group). (FIG. 5E) Intrathecal CSF1 induced microglial proliferation. Inset: BrdU and Ibal colocalization in microglia. Overlap of BrdU and Ibal in inset confirms that the proliferating cells are microglia. Scale bar=100 μm. (FIG. 5F) SNI-induced microglial proliferation (2 day post SNI) persists in DAP12 ko mice (n=3-4 mice/group). (FIG. 5G) Intrathecal CSF1-induced microglia proliferation persists in DAP12 ko mice; (n=3-4 mice/group). Student's t-test or 2-way ANOVA, Tukey's posthoc analysis, p≤0.05, ** p≤0.01, *** p≤0.001, **** p≤0.0001

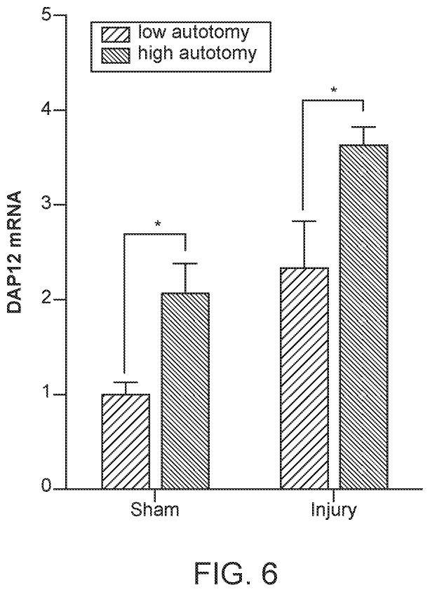

RNA-Seq analysis of the dorsal spinal cord ipsilateral to the nerve injury found a significant upregulation of Tyrobp, the gene that encodes DAP12 (TABLE 2). DAP12 was focused on because it is central to adult microglial functionality (Salter and Beggs, Cell. 2014, 158:15-24; Hickman et al., Nat. Neurosci. 2013, 16:1896-1905) and is induced in microglia in the XIIth nucleus after hypoglossal nerve injury (Kobayashi et al., Glia. 2015, 1073-1082). It is concluded that DAP12 lies downstream of CSF1R and is necessary for the CSF1-CSFR1 triggered upregulation of pain-related microglial genes and of the consequent neuropathic pain condition. Interestingly, DAP12 is also required for hypoglossal nerve injury-induced expression of pro-inflammatory cytokines, including M1-phenotype markers (Kobayashi et al., Glia. 2015, 1073-1082). In the rat, DAP12 mechanisms also contribute to ongoing neuropathic pain. Autotomy (self-mutilation of a denervated limb) is presumed to be driven by a persistent pain comparable to phantom limb pain after amputation. Basal levels of spinal cord DAP12 mRNA were found to be significantly higher in a strain of rats with high autotomy (HA) scores (Devor and Raber, Pain. 1990, 42:51-67) than are DAP12 levels in rats that rarely develop this condition (low autotomy; LA). These DAP12 differences were present both before and after nerve injury (FIG. 6).

FIG. 6: Rats with a predisposition for developing nerve injury-induced autotomy (HA strain) have elevated DAP12 mRNA levels in the spinal cord compared a low autotomy (LA) strain. The differences in DAP12 mRNA levels persist 30 days after injury (n=4 rats/group). Student's ttest, p<0.05.

### Example 5: Microglia Self-Renewal, Rather than Monocyte Infiltration, Underlies Microglial Expansion in the Spinal Cord after Nerve Injury

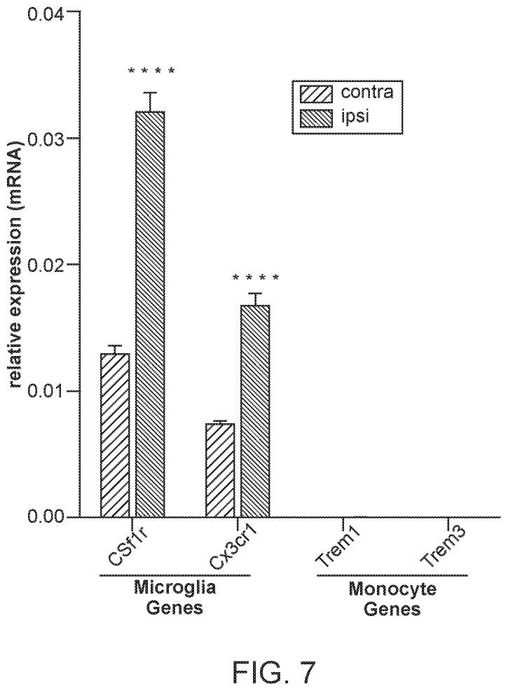

In addition to establishing the neuropathic pain condition, peripheral nerve injury expands the spinal cord microglia population. Despite the comparable gene profile of microglia and monocytes, some genes (Csf1r and Cx3cr1) are expressed at higher levels in microglia; others (Trem1 and Trem3) are expressed exclusively in monocytes (Bedard et al., Glia. 2007, 55:777-789). RNA-Seq analysis showed that although the microglia-enriched genes are upregulated, the monocyte specific genes remained undetectable after nerve injury (TABLE 2). These RNA-Seq findings were confirmed by qRT-PCR (FIG. 7).

FIG. 7: qRT-PCR illustrates that microglia-enriched genes are induced in the ipsilateral dorsal cord 3d after nerve injury; the levels of monocyte specific genes remain undetectable. (n=3 mice/group).

### Example 6: CSF1 is Both Necessary and Sufficient for Nerve Injury-Induced Microglia Proliferation/Self-Renewal in the Dorsal Horn

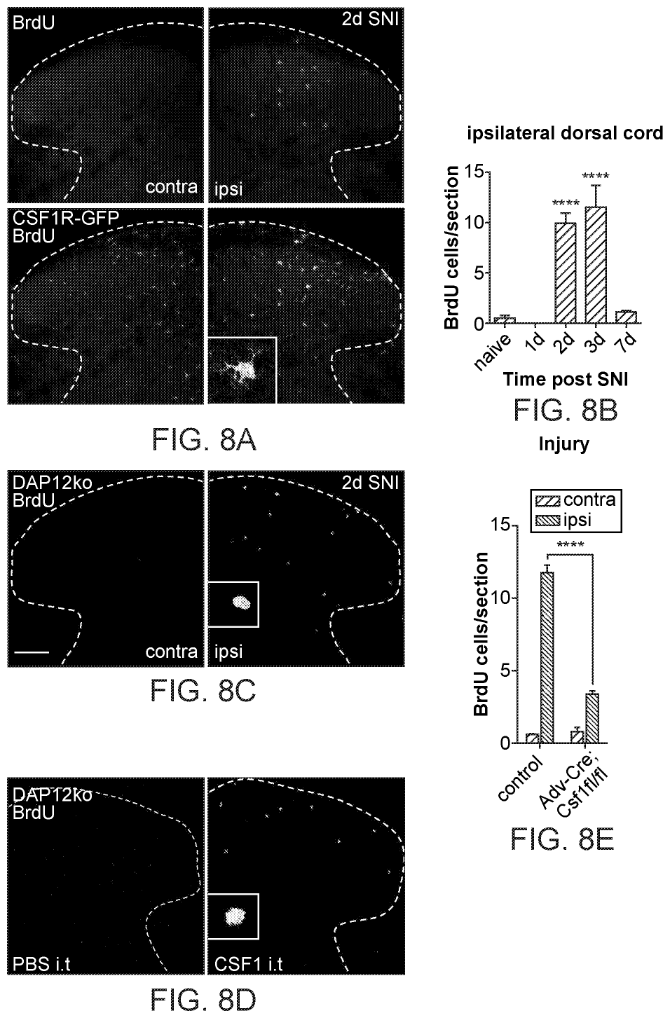

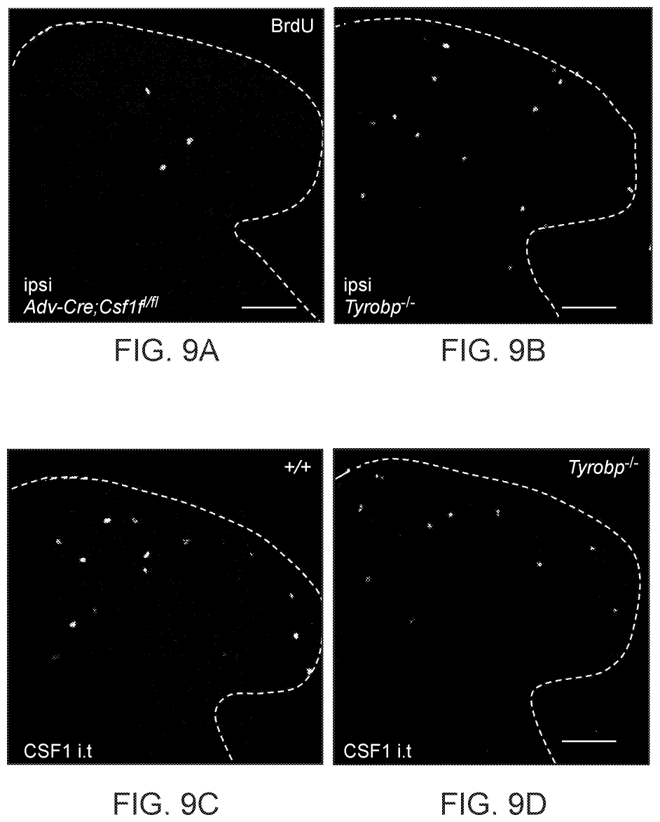

It was next examined whether the de novo expression of CSF1 in injured sensory neurons is also required for nerve injury-induced microglia self-renewal in vivo. A previous report (Echeverry et al., Pain. 2008, 135:37-47) that nerve injury triggers dorsal horn microglia proliferation, was confirmed and demonstrated by incorporation of the thymidine analogue BrdU into CSF1R-expressing microglia (FIG. 8A). Three days following nerve injury, all dorsal horn BrdU+ cells expressed CSF1R, demonstrating that these proliferating cells originate from resident microglia, i.e., the proliferation reflects microglial self-renewal. No microglia proliferation in the dorsal horn was detected at 1 day post injury (FIG. 8B), when CSF1 induction in sensory neurons is readily observed (FIG. 8C; FIG. 2B; FIG. 2D). Advillin-Cre-mediated deletion of Csf1 from DRG neurons largely eliminated the nerve injury-induced dorsal horn microglia proliferation (FIG. 8C; FIG. 9A) Finally, intrathecal injection of CSF1 also induced microglia proliferation in the dorsal horn (FIG. 5F; FIG. 9C), comparable to that provoked by nerve injury (FIG. 8A-8B).

FIG. 8: (FIG. 8A) Double labeling for BrdU and GFP in the CSF1R-GFP mouse shows that BrdU incorporation 2 days after nerve injury is limited to CSF1R-expressing microglia. Inset: Microglial cell double-labeled for BrdU and GFP. (FIG. 8B) Time course of BrdU incorporation after nerve injury. Proliferation begins 2 days after injury and returns to baseline at 1 week (n=3-4 mice/group, two-way ANOVA, Tukey's posthoc analysis. Increase compared to BrdU number in naïve mice). Note that there is no microglial proliferation 1 day after nerve injury, indicating that the upregulation of genes at this time point occurs in resident microglia. (FIG. 8C) Preserved nerve injury-induced microglial proliferation in DAP12 ko mice (2 days post SNI). (FIG. 8D) Preserved intrathecal CSF1-induced (30 ng daily, 3 day) microglial proliferation in DAP12 ko mice. Scale bar=100 μm (FIG. 8E) Advillin-Cre-mediated deletion of Csf1 from sensory neurons significantly decreases injury-induced dorsal horn microglia proliferation (3d post injury, n=3 mice/group)

FIG. 9: (FIG. 9A) Advillin-Cre-mediated deletion of Csf1 from sensory neurons decreases injury-induced dorsal horn microglia proliferation (3d post injury; 3 mice per group); (FIG. 9B) Microglial proliferation 3d post injury persists in Tyrobp^{-/-} mice (3d post injury, n=4 mice); (FIG. 9C) Dorsal horn microglia proliferation after intrathecal CSF1 (3d); (FIG. 9D) Intrathecal CSF1-induced microglia proliferation persists in Tyrobp^{-/-} mice. Scale bar: 100 μm.

### Example 7: DAP12 is not Required for Nerve Injury- or CSF1-Induced Microglia Proliferation In Vivo

The data presented in FIG. 8A-8B show that nerve injury also triggers microglia proliferation, demonstrated by BrdU incorporation into CSF1R expressing microglia, but only beginning 2 days post SNI. It was found that intrathecal CSF1 induced microglia proliferation comparable to that provoked by nerve injury (FIG. 5E). However, neither the microglial proliferation produced by nerve injury nor that produced by intrathecal CSF1 was altered in the DAP12 mutant mice (FIG. 5F-5G; FIG. 8C-8D). Thus, both CSF1 and DAP12 contribute to the rapid neuropathic pain-related gene induction in microglia produced following nerve injury, but only CSF1 contributes to microglia proliferation.

The studies in the SNI model focused on the hypersensitivity that is characteristic of neuropathic pain, but the same mechanisms may contribute to spontaneous, ongoing neuropathic pain. Autotomy (self-mutilation of a denervated limb) is presumed to be driven by ongoing neuropathic pain comparable to the phantom limb pain that occurs after amputation. Interestingly, basal levels of spinal cord DAP12 mRNA are significantly higher in a strain of rats that has a much higher incidence of autotomy than are DAP12 levels in a strain that rarely develops this condition (FIG. 6). The strain differences in DAP12 levels were present both before and after nerve injury.

### Example 8: CSF1 is Induced in Injured Motoneurons and Transported to the Periphery and is Required for Nerve Injury-Induced Microglia Activation and Proliferation in the Ventral Horn

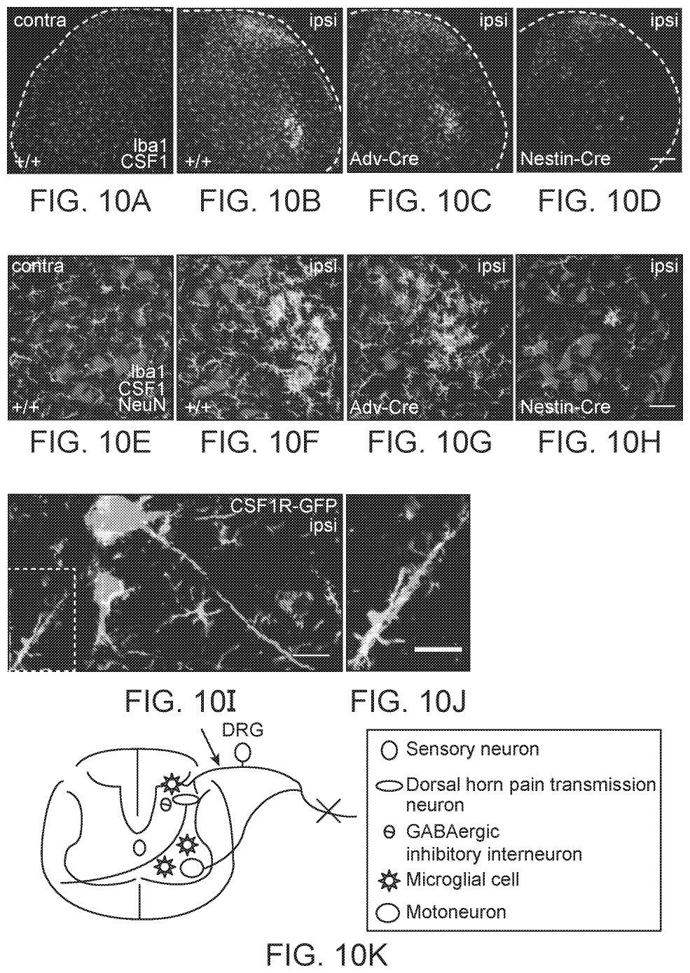

It was also found that peripheral nerve injury induces microglial activation in the ventral horn (around motoneurons; FIG. 10F). Compared to the ventral horn contralateral to the injury (FIG. 10A; FIG. 10E) microglial activation ipsilateral to the injury occurred in close association with de novo CSF1 induction in injured motoneurons (FIG. 10B; FIG. 10F). As in DRG sensory neurons, CSF1 was induced only in injured motoneurons that expressed ATF3.

Although advillin-Cre-mediated sensory neuron deletion of Csf1 greatly reduced nerve injury-induced microglial activation in the dorsal horn (FIG. 10C), the ventral horn microglial activation and the CSF1 induction in motoneurons was only slightly reduced (FIG. 10C; FIG. 10G).

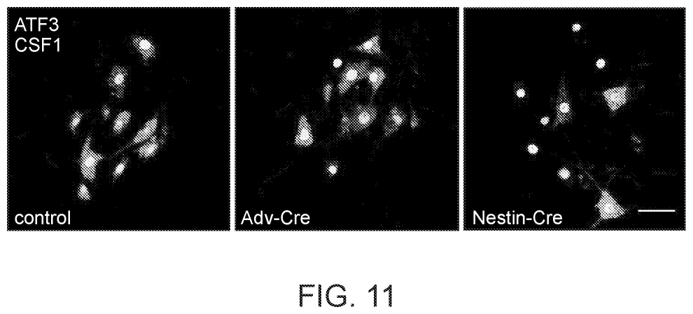

In contrast, nestin-Cre-mediated deletion of Csf1 from the majority (approximately 70%) of CNS motoneurons (FIG. 11) largely eliminated the microglial activation surrounding motoneurons (FIG. 10D; FIG. 10H), without affecting their expression of ATF3 (FIG. 11). In these mice microglial engulfment of motoneurons was also detectable, but only in the residual population of CSF1-expressing motoneurons (FIG. 10D; FIG. 10H).

FIG. 10: (FIG. 10A-10B) CSF1 induction in ventral cord and microglial activation (Ibal) in ventral and dorsal horn 8 days post SNI (control mice) (FIG. 10E-10F) CSF1 expressing motoneurons attract microglia, enlargement of (FIG. 10A-10B); (FIG. 10C) Although specific deletion of CSF1 in DRG neurons prevents microglia activation (Ibal) in the dorsal horn, CSF1 induction in motoneurons is intact and microglial activation around CSF1 expressing motoneurons is preserved (FIG. 10G) Enlargement of ventral cord of (FIG. 10C); (FIG. 10D) CSF1 deletion in the majority of CNS neurons greatly reduces nerve injury induced ventral horn microglia activation, while dorsal horn microglia activation is preserved. (FIG. 10H) Note that remaining CSF1 expressing motoneurons attract microglia. (FIG. 10E-10J) Transport of CSF1 in motoneuron axons. Note close apposition of microglia and CSF1-expressing motoneurons and their axons (arrows). Scale bar=100 μm (FIG. 10H) 50 μm (FIG. 10J).

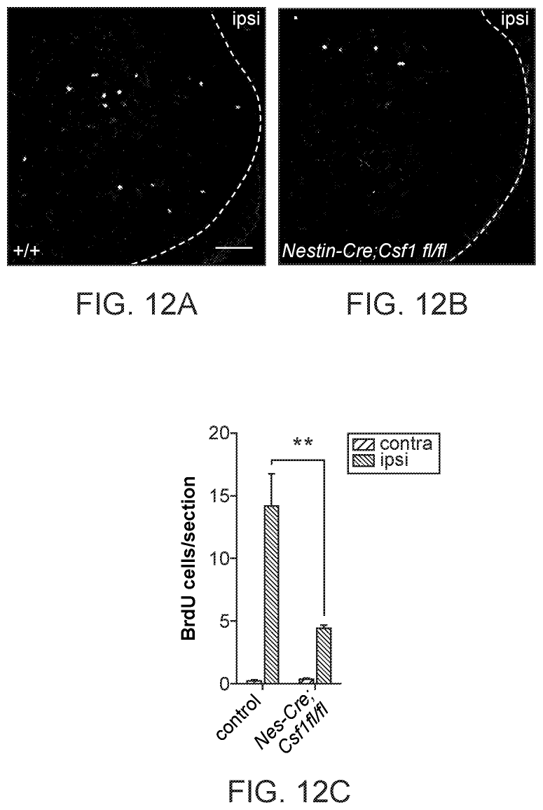

Nerve injury-induced ATF3 expression in axotomized motoneurons was not affected in these mice, but the CSF1 upregulation in motoneurons was significantly reduced (FIG. 11). Only ˜30% of ATF3+ motoneurons expressed CSF1 (FIG. 11), compared to 100% of ATF3+ motoneurons in wild type mice (FIG. 11). The residual expression of CSF1 in motoneurons presumably reflects incomplete Nestin-Cre-mediated recombination in motoneurons. Preventing CSF1 upregulation in motoneurons largely eliminated the nerve injury-induced microglia activation (FIG. 10H) and proliferation (FIG. 12) in the ventral horn.

FIG. 11: In control, Csf1 fl/fl mice, all ATF3-expressing (injured) ventral horn motoneurons coexpress CSF1 after peripheral nerve injury. This pattern does not change significantly in Adv-Cre; Csf1 fl/fl mice. However, in nestin-Cre; Csf1 fl/fl mice, in which Csf1 is deleted from the majority of CNS neurons, ˜30% of ATF3-expressing motoneurons co-express CSF1 after nerve injury. Scale bar: 50 μm.

FIG. 12: (FIG. 12A) Csf1 deletion from the majority of CNS neurons (Nestin-Cre; Csf1 fl/fl) reduces ventral horn microglia activation after injury. Scale bar: 200 μm; (FIG. 12B) Peripheral nerve injury (3d) induces microglia proliferation in the ventral horn, and this is greatly attenuated when Csf1 is deleted from CNS neurons (Nestin-Cre; Csf1 fl/fl). (FIG. 12C) Quantification of (FIG. 12B) and (FIG. 12C); (n=3-4 mice/group).

Finally, in addition to the de novo expression of CSF1 in motoneuron cell bodies and dendrites, and their engulfment by microglia, there is peripheral axonal transport of the CSF1 (FIG. 10E; FIG. 10EJ), presumably to the site of injury. Thus CSF1 appears to contribute to the microglial invasion of motoneuron pools after injury and presumably also to the pathophysiological stripping of their synaptic inputs.

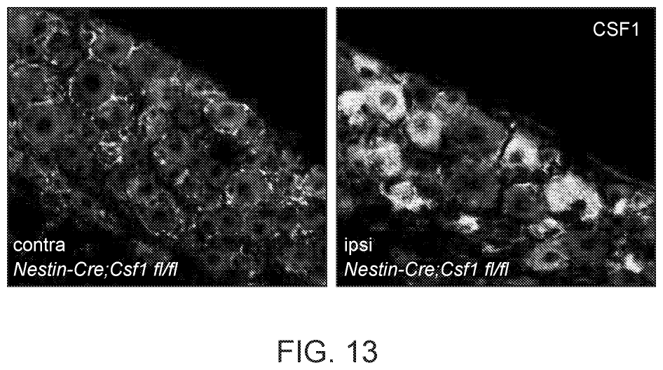

The topographic consequences of neuronal deletion of Csf1 was impressive. Deletion of Csf1 from sensory neurons (Adv-Cre, FIG. 2F) altered neither motoneuronal CSF1 induction nor ventral horn microglial activation after nerve injury (FIG. 10A-10C). Rather, the reduced nerve injury-induced microglia activation was limited to the dorsal horn, within the terminal field of the injured afferents (FIG. 10A-10C). Deletion of Csf1 from CNS neurons (Nestin-Cre, FIG. 11) markedly reduced nerve injury-induced microglia activation in the ventral horn (FIG. 10D). Note that baseline microglial density was also reduced in these mice (FIG. 10). Despite this overall reduction, in these mice the nerve injury-induced CSF1 induction was preserved in sensory neurons (FIG. 13), as was the dorsal horn microglial activation (FIG. 10D).

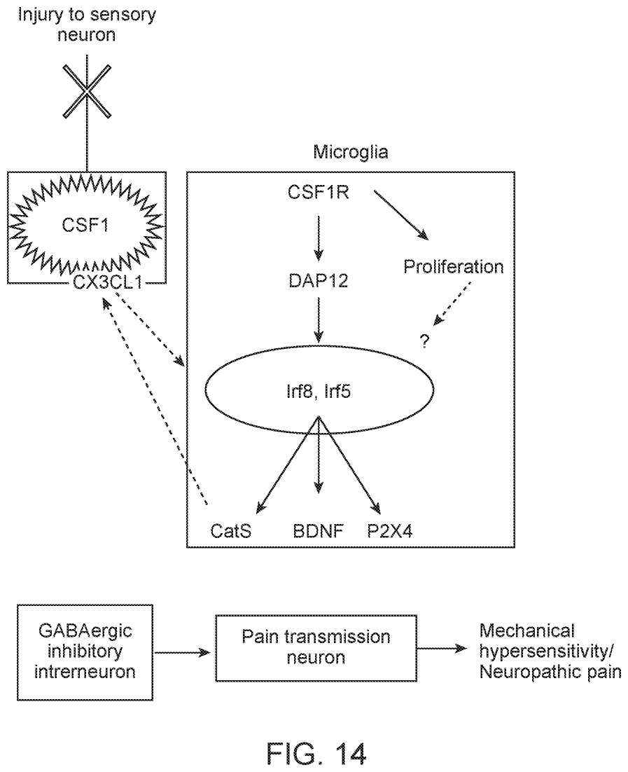

FIG. 14 schematizes the present inventors' findings that link nerve injury-induced changes in sensory neurons with the microglial signaling pathway that influences spinal cord pain transmission circuits. The process begins with the de novo expression of CSF1 in injured sensory neurons. CSF1, in turn, triggers a DAP12-dependent induction of microglial genes, the products of which contribute to the neuropathic pain phenotype, in part by decreasing GABAergic inhibitory controls.

FIG. 13: CSF1 induction in DRG neurons ipsilateral to the nerve injury is preserved in Nestin-Cre; Csf1 fl/fl mice (8d post injury), indicating that Nestin-Cre is not expressed in DRG neurons. Scale bar: 50 μm.

FIG. 14: Within one day of sciatic nerve injury, there is de novo expression of CSF1 in injured (ATF3-positive) DRG sensory neurons. The CSF1 is transported to the spinal cord, where it interacts with microglial CSF1R. Stimulated microglia, in turn, undergo a relatively rapid neuropathic pain-associated gene induction phase (1 day after injury) and a delayed proliferation phase (2 days after injury). Via the DAP12 membrane adaptor protein, CSF1 stimulates microglia to upregulate genes that result in the mechanical hypersensitivity characteristic of neuropathic pain. This DAP12-dependent microglial gene induction likely starts with the upregulation of transcription factors Irf8 and Irf5, which in turn induce the expression of downstream genes, including cathepsin S, BDNF and P2X4. Other studies demonstrated that microglial-derived BDNF ultimately reduces the inhibitory control exerted by GABAergic interneurons, which underlies the hyperexcitability of dorsal horn pain transmission neurons. By an action on neuronal cell membranes, cathepsin S cleaves fractalkine (CX3CL1), which subsequently binds its receptor (CX3CR1) on microglia to amplify the activation of microglia. Through an unidentified DAP12-independent pathway, CSF1 also stimulates microglial proliferation, which contributes to the maintenance of neuropathic pain behavior.

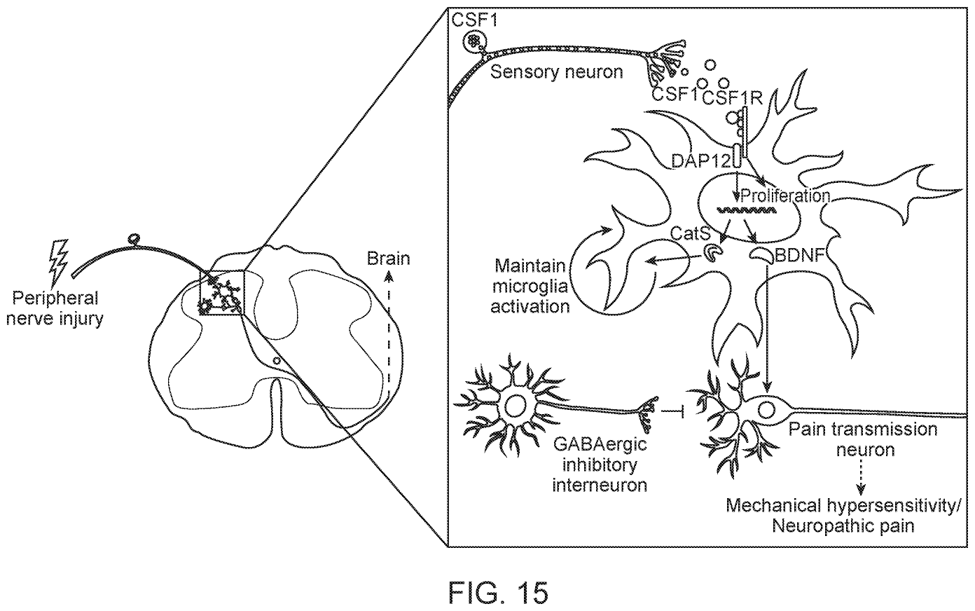

FIG. 15: CSF1 is induced in injured (ATF3-positive) sensory neurons within 1 d of injury and is transported to the spinal cord, where it interacts with microglial CSF1R. Stimulated microglia, in turn, undergo a DAP12-independent proliferation/self-renewal and a DAP12-dependent neuropathic pain-associated gene induction, including BDNF and cathepsin S (CatS). The microglial-derived BDNF contributes to reduced GABAergic inhibitory control and a consequent hyperexcitability of dorsal horn pain transmission neurons. By cleaving CX3CL1 (fractalkine) from neuronal cell membranes, cathepsin S amplifies the activation of microglia. Whether the neuropathic pain phenotype is exacerbated by the concurrent CSF1-induced microglia self-renewal/proliferation and whether DAP12 contributes to that process remains to be determined.

### Example 9: CSF1-Induced Hypersensitivity Involves Microglial Activation and does not Require P2X4

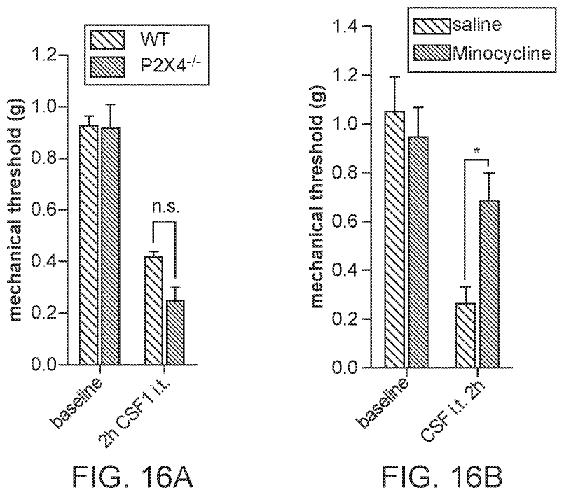

As discussed above, intrathecal administration of CSF1 induced a small but statistically significant increase of Ibal expression. Consistent with these findings, and as shown in FIG. 16A, minocycline prevented the hypersensitivity produced by intrathecal injection of CSF1.

Although the P2X4 receptor is considered critical to the hypersensitivity following nerve injury, intrathecal CSF1-induced mechanical hypersensitivity persisted in mice with a P2X4 knockout, indicating that the CSF1 effect does not require the P2X4 target. The data are shown in FIG. 16B.

In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

1. A polynucleotide comprising a trigeminal ganglion (TGG) or dorsal root ganglion (DRG) promoter operably linked to a recombinant nucleic acid encoding an endonuclease that binds to a nucleotide sequence in the human colony stimulating factor 1 (hCSF1) gene.

2. The polynucleotide of claim 1, wherein binding of the endonuclease to the nucleotide sequence decreases, reduces, or eliminates hCSF gene expression in a dorsal root ganglion cell.

3. The polynucleotide of claim 1, wherein the TGG or DRG promoter is selected from the group consisting of: an hSYN1 promoter, a TRPV1 promoter, a Nav1.7 promoter, a Nav1.8 promoter, a Nav1.9 promoter, a CAG promoter, and an Advillin promoter.

4. The polynucleotide of any of claims 1-3, wherein the nucleotide sequence in the hCSF1 gene is selected from the group consisting of: an hCSF1 gene regulatory region, an hCSF1 promoter, an hCSF1 transcription start site, an hCSF1 exon sequence, an hCSF1 intronic sequence, and an hCSF1 5′ or 3′ untranslated region.

5. The polynucleotide of claim 1, wherein the endonuclease is an endonuclease that is engineered to bind the nucleotide sequence of the hCSF1 gene.

6. The polynucleotide of claim 5, wherein the engineered endonuclease is a homing endonuclease, a transcription activator-like effector nucleases (TALENs), a zinc finger nuclease (ZFN), a Type II clustered regularly interspaced short palindromic repeats (CRISPR) associated (Cas9) nuclease, or a megaTAL nuclease.

7. The polynucleotide of claim 6, wherein the homing endonuclease is a LAGLIDADG endonuclease, a GIY-YIG endonuclease, a His-Cys box endonuclease, or an HNH endonuclease.

8. The polynucleotide of claim 6, wherein the homing endonuclease is I-Onu I, I HjeMI, I-CpaMI, I-Sce I, I-Chu I, I-Dmo I, I-Cre I, I-Csm I, PI-Sce I, PI-T11 I, PI-Mtu I, I-Ceu I, I-Sce II, I-Sce III, HO, P1-Civ I, PI-Ctr I, PI-Aae I, PI-Bsu I, PI-Dha I, PI-Dra I, PI-May I, PI-Mch I, PI-Mfu I, PI-Mfl I, PI-Mga I, PI-Mgo I, PI-Min I, PI-Mka I, PI-Mle I, PI-Mma I, PI-Msh I, PI-Msm I, PI-Mth I, PI-Mtu I, PI-Mxe I, PI-Npu I, PI-Pfu I, PI-Rma I, PI-Spb I, PI-Ssp I, PI-Fac I, PI-Mja I, PI-Pho I, PI-Tag I, PI-Thy I, PI-Tko I, or PI-Tsp I.

9. The polynucleotide of claim 6, wherein the Cas9 nuclease is from Streptococcus pyogenes, Streptococcus thermophilus, Treponema denticola, or Neisseria meningitidis.

10. The polynucleotide of claim 9, wherein the Cas9 nuclease comprises one or more mutations in a HNH or a RuvC-like endonuclease domain or the HNH and the RuvC-like endonuclease domains.

11. The polynucleotide of claim 10, wherein the mutant Cas9 nuclease is a nickase.

12. The polynucleotide of any one of the preceding claims, wherein the polynucleotide further comprises a RNA polymerase III promoter operably linked to a crRNA and a tracrRNA or to a single guide RNA (sgRNA).

13. The polynucleotide of claim 12, wherein the RNA polymerase III promoter is the human or mouse U6 snRNA promoter, the human or mouse H1 RNA promoter, or the human tRNA-val promoter.

14. The polynucleotide of claim 11, wherein the polynucleotide comprises a pair of offset crRNAs or sgRNAs.

15. The polynucleotide of any one of claims 12-14, wherein the pair of crRNA or sgRNAs are offset by about 25 to about 100 nucleotides from each other.

16. The polynucleotide of any of the preceding claims, wherein the endonuclease comprises a TREX2 domain.

17. A polynucleotide comprising a promoter operable in a TGG or DRG that is operably linked to an inhibitory RNA that binds to an hCSF1 mRNA.

18. The polynucleotide of claim 17, wherein the TGG or DRG promoter is an inducible promoter.

19. The polynucleotide of claim 18, wherein the inducible promoter comprises a tetracycline inducible promoter, a LOX-stop-LOX human or mouse U6 snRNA promoter, LOX-stop-LOX human or mouse H1 RNA promoter, or a LOX-stop-LOX human tRNA-val promoter.

20. The polynucleotide of claim 17, wherein the TGG or DRG promoter is selected from the group consisting of: an hSYN1 promoter, a TRPV1 promoter, a Nav1.7 promoter, a Nav1.8 promoter, a Nav1.9 promoter, a CAG promoter, and an Advillin promoter.

21. The polynucleotide of claim 17, wherein the polynucleotide comprises a TGG or DRG promoter operably linked to a Cre recombinase and a LOX-stop-LOX inducible RNA polymerase III promoter operably linked to the inhibitory RNA.

22. The polynucleotide of any one of claims 17-21, wherein the inhibitory RNA is an siRNA, an miRNA, an shRNA, a ribozyme, or a piRNA.

23. A vector comprising the polynucleotide of any one of claims 1-22.

24. The vector of claim 23, wherein the vector is a plasmid-based vector or a viral vector.

25. The vector of claim 23 or claim 24, wherein the vector is episomal or non-integrative.

26. The vector of claim 25, wherein the viral vector is retroviral vector, an adenoviral vector, an adeno-associated viral (AAV) vector or a herpes simplex virus (HSV) vector.

27. The vector of claim 26, wherein the retroviral vector is a lentiviral vector or a gamma retroviral vector.

28. The vector of claim 26, wherein the AAV comprises a serotype selected from the group consisting of: AAV9, AAV6, AAVrh10, AAV7M8, and AAV24YF.

29. The vector of claim 25, wherein the HSV vector comprises a serotype selected from the group consisting of: JΔNI5, JΔNI7, and JΔNI8.

30. A vector comprising a polynucleotide comprising an hSYN1 promoter operably linked to a nucleic acid encoding a Cas9 nuclease and a polynucleotide comprising an U6 RNA polymerase III promoter operably linked to an hCSF1 gene targeted sgRNA.

31. A method of treating neuropathic pain comprising administering a subject in need thereof, a vector according to any one of claims 23-30.

32. A method of providing analgesia to a subject comprising administering to the subject, a vector according to any one of claims 23-30.

33. A method of decreasing hCSF1 expression in a TGG or DGG of a subject, comprising administering to the subject, a vector according to any one of claims 23-30.

34. A method of reducing nerve injury induced mechanical hypersensitivity and microglia activation comprising administering to the subject, a vector according to any one of claims 23-30.

35. The method of any one of claims 31-34, wherein the vector is administered to the subject by intrathecal bolus injection or infusion, intraganglionic injection, intraneural injection, subcutaneous injection, or intraventricular injection.

36. The method of claim 35, wherein the vector is administered to the subject by intrathecal bolus injection or infusion at multiple levels of the spinal column for DRG transduction.

37. The method of claim 35, wherein the vector is administered to the subject by intraganglionic injection directly into a single dorsal root ganglion, multiple dorsal root ganglia, or the trigeminal ganglion.

38. The method of claim 35, wherein the vector is administered to the subject by intraneural injection into the nerve bundle (e.g. sciatic nerve, trigeminal nerve).

39. The method of claim 35, wherein the vector is administered to the subject by subcutaneous injection at the peripheral nerve terminals (subdermal or internal organ wall).

40. The method of claim 35, wherein the vector is administered to the subject by intraventricular injection (for trigeminal ganglion transduction).


SEQUENCE LISTING (34 sequences)

SEQ ID NO: 1 - DNA, 20 bp, Artificial sequence
  Synthetic nucleotide sequence
tgctaagtgctctagccgag

SEQ ID NO: 2 - DNA, 20 bp, Artificial sequence
  Synthetic nucleotide sequence
cccccaacagtcagcaagac

SEQ ID NO: 3 - DNA, 20 bp, Artificial sequence
  Synthetic nucleotide sequence
ccacacccgccaccagttcg

SEQ ID NO: 4 - DNA, 20 bp, Artificial sequence
  Synthetic nucleotide sequence
tacagcccggggagcatcgt

SEQ ID NO: 5 - DNA, 19 bp, Artificial sequence
  Synthetic nucleotide sequence
acgtacagcggagcctcat

SEQ ID NO: 6 - DNA, 20 bp, Artificial sequence
  Synthetic nucleotide sequence
catgacccggaagcagttgt

SEQ ID NO: 7 - DNA, 21 bp, Artificial sequence
  Synthetic nucleotide sequence
ccgaggtcaagggacagcgga

SEQ ID NO: 8 - DNA, 25 bp, Artificial sequence
  Synthetic nucleotide sequence
tgcctctgtgtgttgaggtcactgt

SEQ ID NO: 9 - DNA, 20 bp, Artificial sequence
  Synthetic nucleotide sequence
gagtctgcctccgtgtccgc

SEQ ID NO: 10 - DNA, 20 bp, Artificial sequence
  Synthetic nucleotide sequence
tacgtgagcggccagggtct

SEQ ID NO: 11 - DNA, 21 bp, Artificial sequence
  Synthetic nucleotide sequence
gcctctggtggagtctgcgtg

SEQ ID NO: 12 - DNA, 23 bp, Artificial sequence
  Synthetic nucleotide sequence
cgcccaaataacaggcctcagca

SEQ ID NO: 13 - DNA, 20 bp, Artificial sequence
  Synthetic nucleotide sequence
caggttcgagaggtctgacg

SEQ ID NO: 14 - DNA, 20 bp, Artificial sequence
  Synthetic nucleotide sequence
aagtgtacaagtccgcgtcc

SEQ ID NO: 15 - DNA, 21 bp, Artificial sequence
  Synthetic nucleotide sequence
gggggcatagaggcagacgct

SEQ ID NO: 16 - DNA, 22 bp, Artificial sequence
  Synthetic nucleotide sequence
gggcatcctcgtcaccaaacgg

SEQ ID NO: 17 - DNA, 20 bp, Artificial sequence
  Synthetic nucleotide sequence
cgactatgtggtcccagctc

SEQ ID NO: 18 - DNA, 20 bp, Artificial sequence
  Synthetic nucleotide sequence
gcgtctgaatcgcaaatgct

SEQ ID NO: 19 - DNA, 16 bp, Artificial sequence
  Synthetic nucleotide sequence
gggcagcgtgggaacc

SEQ ID NO: 20 - DNA, 20 bp, Artificial sequence
  Synthetic nucleotide sequence
gcttccaggggatacggaac

SEQ ID NO: 21 - DNA, 20 bp, Artificial sequence
  Synthetic nucleotide sequence
tggggacaacaccatcttca

SEQ ID NO: 22 - DNA, 20 bp, Artificial sequence
  Synthetic nucleotide sequence
ctggaagtcacggcttttgt

SEQ ID NO: 23 - DNA, 20 bp, Artificial sequence
  Synthetic nucleotide sequence
aaacagcacatggctgagac

SEQ ID NO: 24 - DNA, 21 bp, Artificial sequence
  Synthetic nucleotide sequence
gcatagggtgggttcatctgt

SEQ ID NO: 25 - DNA, 21 bp, Artificial sequence
  Synthetic nucleotide sequence
accactgacttgctggccccg

SEQ ID NO: 26 - DNA, 21 bp, Artificial sequence
  Synthetic nucleotide sequence
cgacgggtgctttccagggac

SEQ ID NO: 27 - DNA, 20 bp, Artificial sequence
  Synthetic nucleotide sequence
acacgcacggccaccatgaa

SEQ ID NO: 28 - DNA, 22 bp, Artificial sequence
  Synthetic nucleotide sequence
gcatggaccgtgaggatgaggc

SEQ ID NO: 29 - DNA, 20 bp, Artificial sequence
  Synthetic nucleotide sequence
actgctgtgcgtgttctttg

SEQ ID NO: 30 - DNA, 20 bp, Artificial sequence
  Synthetic nucleotide sequence
gccttctggctgttggcata

SEQ ID NO: 31 - DNA, 20 bp, Artificial sequence
  Synthetic nucleotide sequence
caagatgtggggctgtacca

SEQ ID NO: 32 - DNA, 20 bp, Artificial sequence
  Synthetic nucleotide sequence
aagccacacgtcagaacgat

SEQ ID NO: 33 - DNA, 21 bp, Artificial sequence
  Synthetic nucleotide sequence
agcggacagcatcctccggag

SEQ ID NO: 34 - DNA, 21 bp, Artificial sequence
  Synthetic nucleotide sequence
gtctgcgtcttcggccatggt